SEQUENCE LISTING (5 sequences)

SEQ ID NO: 1 - protein, 191 aa, homo sapiens [sequence omitted]

SEQ ID NO: 2 - protein, 620 aa, homo sapiens
FSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 3 - protein, 227 aa, homo sapiens
  MISC_FEATURE (1)..(10): hinge region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 225 aa, Rattus rattus
  MISC_FEATURE (1)..(10): hinge region
VPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGK

SEQ ID NO: 5 - protein, 31 aa, Artificial Sequence
  Synthetic sequence
SGGGGSGGGGSGGGGSGGGGSGGGGSGGGGS

(19) United States
(12) Patent Application Publication
(10) Pub. No.: US 2016/0017017 A1
(43) Pub. Date: Jan. 21, 2016

(54) Growth Hormone Compounds

(71) Applicants: Xin ZHAO, Beijing (CN); Peter THYGESEN, Bagsværd (DK); Nils Langeland JOHANSEN, Bagsværd (DK); Leif NØRSKOV-LAURITSEN, Bagsværd (DK); NOVO NORDISK HEALTH CARE AG, Zürich (CH)

(72) Inventors: Xin Zhao, Beijing (CN); Peter Thygesen, Koebenhavn OE (DK); Nils Langeland Johansen, Koebenhavn OE (DK); Leif Noerskov-Lauritsen, Tappernoeje (DK)

(73) Assignee: NOVO NORDISK HEALTHCARE AG, Zurich (CH)

(21) Appl. No.: 14/773,433

(22) PCT Filed: Mar. 11, 2014
(86) PCT No.: PCT/EP2014/054669

§ 371 Date: Sep. 8, 2015

Related U.S. Application Data
(60) Provisional application No. 61/811,917, filed on Apr. 15, 2013.

(30) Foreign Application Priority Data
Mar. 11, 2013 (CN) PCT/CN2013/072383
Apr. 12, 2013 (EP) 13163478.4

(51) Int. Cl. C07K 14/61

(52) U.S. Cl. CPC C07K 14/61; C07K 2319/30; A61K 38/00

(57) ABSTRACT

The invention relates to growth hormone compounds with a long plasma half-life obtained by Fc linkage. An increased half-life is an advantage allowing a less frequent or low dosage administration of therapeutic. The invention further relates to methods of producing such compound including expression vectors for heterologous expression.

### TECHNICAL FIELD

The field of the present invention is growth hormone compounds mainly prepared by recombinant methods. As wild type growth hormone such compounds are useful for treatment of diseases or disorders where an increased plasma concentration of a compound with growth hormone activities is beneficial to the patient.

### BACKGROUND

Growth hormone (GH) is a polypeptide hormone secreted by the anterior pituitary in mammals. Dependent on species GH is a protein composed of approximately 190 amino acid residues corresponding to a molecular weight of approximately 22 kDa. GH binds to and signals through cell surface receptors, the GH receptors (GHR). GH plays a key role in promoting growth, maintaining normal body composition, anabolism and lipid metabolism. It also has direct effects on intermediate metabolism, such as decreased glucose uptake, increased lipolysis, increased amino acid uptake and protein synthesis. The hormone also exerts effects on other tissues including adipose tissue, liver, intestine, kidney, skeleton, connective tissue and muscle. Recombinant hGH (somatropin) is commercially available as, for ex: Genotropin™ (Pfizer), Nutropin™ and Protropin™ (Genentech), Humatrope™ (Eli Lilly), Serostim™ (Serono), Norditropin (Novo Nordisk), Omnitrope (Sandoz), Nutropin Depot (Genentech and Alkermes). Additionally, an analogue with an additional methionine residue at the N-terminal end is also marketed as, for ex: Somatonorm™ (Pharmacia Upjohn/Pfizer).

GH shares a common topology with the other members of the GH-family of proteins, Prolactin (PRL) and Placental Lactogen (PL). GH is classified as a four-helix bundle protein exhibiting an “up-up-down-down” topology with two conserved disulphide linkages. Specifically, the mature wild-type human GH (hGH identified by SEQ ID NO: 1) is composed of 191 amino acid residues and has four cysteine residues at positions 53, 165, 182 and 189, which stabilizes the three dimensional structure of the protein by forming two intramolecular disulphide bonds connecting C53 with C165 and C182 with C189, respectively. The structure of hGH has been experimentally determined by X-ray crystallography in the free form (Chantalet L. et al (1995) Protein and Peptide Letters 3, 333-340) and in complex with its binding protein (the extra cellular domain of the human GHR (hGHR)) (deVos, A. M. et al (1992) Science 255, 306-312). These structures have been deposited in the Protein Data Bank (PDB) and are publicly available (PDB accession codes 1 HGU and 1 HWG, respectively). Thus, from the published hGH structures residues important for hGH binding to hGHR can be identified. Studies have demonstrated that various mutants have lower affinity to the growth hormone receptor (Cunningham B C, Proc Natl Acad Sci USA. 1991 Apr. 15; 88(8):3407-11 and Cunningham B C, Wells J A, Science. 1989 Jun. 2; 244(4908):1081-5). Furthermore, the dynamic properties of hGH has been studied by Nuclear Magnetic Resonance (NMOLR) spectroscopy (Kasimova M. R. et al. J. Mol. Biol. (2002) 318, 679-695). The mature human GHR is identified herein by SEQ ID NO: 2 including AA 19-638 of the protein coded by the GHR gene resulting from the removal of the signal peptide. The extra cellular part also referred to the growth hormone binding protein constitutes AA 19-256.

hGH has been subject to extensive mutagenesis and modified in attempts to produce hGH analogues and conjugates hereof with altered chemical or biological properties including cysteine mutants, protease stabilized mutants and PEGylated versions of growth hormone as described in such as US 2003/0162949, WO 02/055532 and WO2006048777.

In the quest for growth hormone compounds with increased functionality compounds with an increased half-life is desirable in order to reduce the amount of compound needed and the frequency of administration of the therapeutic.

Inspired by the huge success of antibody treatment intensive research in use of an antibody immunoglobulin Fc region as protractor has provided growth hormone compounds with an increased half-life such as the compounds described in WO2012/008779 and WO2006/107124, WO2005 047334/35/36/37 where the Fc region is covalently linked to growth hormone via a non-peptidyl polymer (PEG), and growth hormone fusions, such as Fc fusions as described in WO 01/03737, WO 200814743, U.S. Pat. No. 7,404,956, WO 04101739 and WO2005001025. US2012/0116056 describes Fc fusion proteins of human growth hormone (hGH) and a selection of specified human IgG Fc variants aimed at reducing undesirable Fc-mediated side effects.

Although such methods are suitable to increase half-life of hGH the wish to increase the half-life and stability of growth hormone compounds even more remains a high priority in order to be able to provide convenient therapeutics to patients.

Sequence listing:

SEQ ID NO: 1: mature hGH 1-191 (Somatotropin) (referred to as
hGH for short)
FPTIPLSRLFDNAMLRAHRLHQLAFDTYQEFEEAYIPKEQKYSFLQNPQTSLCFSESIPTPSN
REETQQKSNLELLRISLLLIQSWLEPVQFLRSVFANSLVYGASDSNVYDLLKDLEEGIQTLMG
RLEDGSPRTGQIFKQTYSKFDTNSHNDDALLKNYGLLYCFRKDMDKVETFLRIVQCRSVEG
SCGF
SEQ ID NO: 2: mature human GHR 19-638 of human GHR
FSGSEATAAILSRAPWSLQSVNPGLKTNSSKEPKFTKCRSPERETFSCHWTDEVHHGTKN
LGPIQLFYTRRNTQEWTQEWKECPDYVSAGENSCYFNSSFTSIWIPYCIKLTSNGGTVDE
KCFSVDEIVQPDPPIALNWTLLNVSLTGIHADIQVRWEAPRNADIQKGWMVLEYELQYKE
VNETKWKMMDPILTTSVPVYSLKVDKEYEVRVRSKQRNSGNYGEFSEVLYVTLPQMSQFT
CEEDFYFPWLLIIIFGIFGLTVMLFVFLFSKQQRIKMLILPPVPVPKIKGIDPDLLKEGK
LEEVNTILAIHDSYKPEFHSDDSWVEFIELDIDEPDEKTEESDTDRLLSSDHEKSHSNLG
VKDGDSGRTSCCEPDILETDFNANDIHEGTSEVAQPQRLKGEADLLCLDQKNQNNSPYHD
ACPATQQPSVIQAEKNKPQPLPTEGAESTHQAAHIQLSNPSSLSNIDFYAQVSDITPAGS
VVLSPGQKNKAGMSQCDMHPEMVSLCQENFLMDNAYFCEADAKKCIPVAPHIKVESHIQP
SLNQEDIYITTESLTTAAGRPGTGEHVPGSEMPVPDYTSIHIVQSPQGLILNATALPLPD
KEFLSSCGYVSTDQLNKIMP
SEQ ID NO 3: human Fc IgG1
DKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
- wherein the underlined sequence refers to the hinge region and
the bolded AA are amino acid residues corresponding to L234,
L235, G237, A330 and P331 in a full-length heavy chain sequence.
SEQ ID NO 4: rat Fc IgG2a
VPRECNPCGCTGSEVSSVFIFPPKTKDVLTITLTPKVTCVVVDISQNDPEVRFSWFIDDVEVH
TAQTHAPEKQSNSTLRSVSELPIVHRDWLNGKTFKCKVNSGAFPAPIEKSISKPEGTPRGPQ
VYTMAPPKEEMTQSQVSITCMVKGFYPPDIYTEWKMNGQPQENYKNTPPTMDTDGSYFLY
SKLNVKKETWQQGNTFTCSVLHEGLHNHHTEKSLSHSPGK
- wherein the underlined sequence refers to the hinge region.
SEQ ID NO 5: GS linker (S(GGGGS)₆)
SGGGGSGGGGSGGGGSGGGGSGGGGSGGGGS

### SUMMARY

The main aspect of the invention relates to growth hormone compounds that have an extended half-life as exemplified herein by a fusion to an immunoglobulin Fc region. The compounds are further characterized by an altered amino acid sequence, changing one or more amino acid residue(s), compared to human growth hormone. The unusual character of such mutation(s) aims to partially alter the functionality compared to human growth hormone.

Such growth hormone variants in the context of a protracted growth hormone compound provides an even further prolonged half-life while retaining biological activity of human growth hormone. Such compound may thus help to solve the problem of providing a therapeutic growth hormone compound that can be administered with low frequency and in low dosages to provide patients with convenient product capable of increasing growth hormone activity in the blood. In one embodiment the invention relates to growth hormone compounds comprising a growth hormone variant linked to an antibody Fc-domain (GH-variant-Fc). In one such embodiment the compound is made up by two polypeptides one being a GH-variant-Fc polypeptide fusion and a Fc-polypeptide linked with the Fc sequence of the GH-variant-Fc polypeptide fusion. In on embodiment the growth hormone compound includes 1 to 5 point mutations in the sequence of GH variant, which reduces affinity to the growth hormone receptor. The growth hormone compounds according to the invention display an extended half-life (T½) in vivo as well as the ability to induce an extended IGF-response.

Further aspects of the invention relates to methods and tools for producing such compounds, in particular, DNA sequences and vectors encoding a GH-variant-Fc fusion polypeptide and host cells comprising such vectors encoding the GH-variant-Fc fusion polypeptide optionally in combination with a vector encoding a Fc polypeptide.

### BRIEF DESCRIPTION OF DRAWINGS

FIG. 1 shows the pharmacokinetic profiles of four growth hormone variant Fc-fusion compounds. Each growth hormone Fc-fusion compound has the same general structure: hGH-Linker-Fc(IgG1)/Fc(IgG1). Mutations in the growth hormone sequence are indicated in the figure legend for each molecule.

FIG. 2 shows, the IGF-1 plasma concentration-time profile of a growth hormone variant Fc-fusion compounds after a single dose i.v. administration to Sprague Dawley rats. FIG. 2A displays the total concentration of IGF-1 whereas FIG. 2B displays the baseline corrected IGF-1 plasma concentration. Mutations in the growth hormone sequence are indicated in the figure legend for each molecule.

FIG. 3 shows, the effect of growth hormone variant Fc-fusion compounds on body weight of hypophysectomised Sprague Dawley rats after a single dose i.v. administration of 15 nmolol. Mutations in the growth hormone seqeunce are indicated in the figure legend for each molecule.

### DEFINITIONS

The term “binding affinity” is herein used as a measure of the strength of a non-covalent interaction between two molecules, e.g. a receptor and a ligand. The term “binding affinity” is used to describe monovalent interactions (intrinsic activity).

The term “nucleotide sequence” is used to define a DNA (or RNA sequence) that encode a polypeptide.

The term “amino acid” includes the group of the amino acids encoded by the genetic code which are herein referred to as standard amino acid. Further included are natural amino acids which are not encoded by the genetic code, as well as synthetic amino acids.

The term “polypeptide” and “peptide” as used herein means a compound composed of at least two amino acids connected by peptide bond(s).

The term “protein” as used herein means a biochemical compound consisting of one or more polypeptides.

The term “drug”, “therapeutic”, “medicament” or “medicine” when used herein refer to an active ingredient used in a pharmaceutical composition, which may be used in therapy.

The term “growth hormone variant” is used herein to describe analogues of a growth hormone protein which has an amino acid sequence which is different from the sequence of mature human growth hormone identified by SEQ ID NO 1.

The term “Fc-domain” is used herein to describe the complex of two Fc polypeptides.

The term “complex” is used herein to describe the association of at least two polypeptides which are not linked by peptide bond(s) established during translation of the encoding nucleotide sequences. The association may be covalent or non-covalent.

The term “protracting moiety” is used here in to define a unit that has ability to prolong in vivo half-life of proteins. The term thus includes well known entities, such as Fc-domains, PEG molecules, serum albumin, albumin binders, the growth hormone binding protein, AA polymers (as used in the XTEN technology (Amunix, Mountain View, Calif.), and used in PASylation® (XLprotein GMBH, Freising, Germany)), and carbohydrate groups such as hydroxyethylstarch and heparosan.

### DESCRIPTION

The present invention relates to growth hormone compounds comprising a growth hormone variant linked to an antibody Fc-domain, such compound may for short be referred to as “GH-variant-Fc”.

### Growth Hormone Compounds

The mature human growth hormone protein is composed of four helixes (Helix 1-4 or H1-4) connected by three loops (Loop 1-3 or L1-3), and a C-terminal segment. In human growth hormone Helix 1 is defined by AA residue 6-35, Helix 2 is defined by AA residues 71-98, Helix 3 is defined by AA residue 107-127 and Helix 4 is defined as AA residues 155-184. By interaction with the growth hormone receptor growth hormone mediates its biological activity.

As will be described herein below, growth hormone compounds according to the present invention is composed of a growth hormone part and a protracting part, the growth hormone part is considered responsible for the biological functionality of the compound whereas the protracting part is at least partly responsible for extending the half-life of the growth hormone compound.

The growth hormone compounds will thus usually include a growth hormone polypeptide e.g. an amino acid chain with a high level of identity to human growth hormone as identified by SEQ ID NO 1. As described herein below the growth hormone compounds according to the present invention includes a growth hormone variant as defined by a protein sequence that differs from the sequence of human growth hormone.

Growth hormone compounds according to the invention encompass both growth hormone fusions and growth hormone conjugates. As evident from the wording growth hormone fusions compounds include a growth hormone sequence linked to a second protein sequence by means of at least one peptide bond, whereas a growth hormone conjugate refers to a covalent linkage of the growth hormone part and the protracting part by way of a chemical modification. Generation of fusion proteins is well known in the art and usually obtained by expression of the fusion protein using a recombinant expression vector linking a DNA sequence encoding said growth hormone sequence with a second DNA sequence encoding said second protein optionally including a linker sequence. Growth hormone fusions include, but are not limited to, fusions comprising an antibody Fc-domain.

Likewise conjugation of the growth hormone part and the protracting part may be obtained by methods know in the art, where the Fc-domain is covalently attached to the growth hormone variant. Attachment may be to the N-terminal or C-terminal amino acid residues or to an in-chain amino acid residue of the growth hormone variant. Point mutations may be introduced in growth hormone to facilitate such attachment of a protracting moiety.

In one embodiment the invention relates to a growth hormone compound comprising a growth hormone variant linked to an antibody Fc-domain, which may be described by GH-variant-Fc.

### Growth Hormone Variant

A growth hormone variant according to the invention has a sequence different from the sequence of mature human growth hormone identified by SEQ ID NO.: 1.

The difference may be caused by addition and/or deletion and/or substitution of at least one amino acid residue compared to the naturally occurring human growth hormone sequence. The term is used for a mutant growth hormone protein wherein one or more amino acid residues of the growth hormone sequence has/have been substituted by another amino acid residues and/or wherein one or more amino acid residues have been deleted from the growth hormone and/or wherein one or more amino acid residues have been added and/or inserted to the growth hormone.

In one embodiment a growth hormone variant according to the invention comprises less than 8 modifications (substitutions, deletions and/or additions) relative to human growth hormone. In one embodiment a growth hormone variant comprises less than 7 modifications (substitutions, deletions and/or additions) relative to human growth hormone. In one embodiment a growth hormone variant comprises less than 6 modifications (substitutions, deletions and/or additions) relative to human growth hormone. In one embodiment a growth hormone variant comprises less than 5 modifications (substitutions, deletions and/or additions) relative to human growth hormone. In one embodiment a growth hormone variant comprises less than 4 modifications (substitutions, deletions and/or additions) relative to human growth hormone. In one embodiment a growth hormone variant comprises less than 3 modifications (substitutions, deletions and/or additions) relative to human growth hormone. In one embodiment a growth hormone variant comprises less than 2 modifications (substitutions, deletions and/or additions) relative to human growth hormone. In a series of embodiment the growth hormone variant of the growth hormone is at least 95, 96, 97, 98 or 99% identical to human growth hormone identified by SEQ ID NO: 1.

In one embodiment a growth hormone variant comprises exactly 7 amino acid modifications. In one embodiment a growth hormone variant comprises exactly 6 amino acid modifications. In one embodiment a growth hormone variant comprises exactly 5 amino acid modifications. In one embodiment a growth hormone variant comprises exactly 4 amino acid modifications. In one embodiment a growth hormone variant comprises exactly 3 amino acid modifications. In one embodiment a growth hormone variant comprises exactly 2 amino acid modifications. In one embodiment a growth hormone variant comprises exactly 1 amino acid modifications.

As described above the overall structure of growth hormone is well known and the second and three dimensional structures identify various regions which may at least partly be allocated different functionalities. The secondary structure defines Helix 1-4 and the intervening Loops 1-3. The growth hormone receptor binding has been allocated to three dimensional sites termed site 1 and site 2 which each span multiple discontinuous amino acid stretches. As described herein the GH compound/GH variants preferably has a lower binding affinity to hGHR via site 1 or site 2 compared to hGH or a GH compound comprising human growth hormone identified by SEQ ID NO 1 due to a point mutation or AA deletion in site 1 or site 2.

In one embodiment the growth hormone variant has at least one point mutation in site 1 (Helix 1(9-35) and Loop 1 (36-71) and Helix 4 (155-184)), such as in Helix1, in positions corresponding to Met14, His18, His21, Gln22 and Phe25.

In one embodiment the growth hormone variant has at least one point mutation in Loop 1, such as in positions corresponding to Lys41, Tyr42, Leu45, Gln46, Asn47, Pro48, Gln49, Ser51, Leu52, Pro59, Pro61, Ser62, Asn63, Arg64, Glu65, Thr67 and Gln68 or such as in positions corresponding to Lys41, Leu45, Pro59, Pro61, Arg64 and Glu65.

In one embodiment the growth hormone variant has at least one point mutation in Helix 4 such as in positions corresponding to Tyr164, Arg167, Lys168, Asp171, Lys172, Glu174, Thr175, Phe176, Arg178, Ile179, Cys182, Cys189 and Gly190 or such as in positions corresponding to Lys172, Glu174, Thr175, Phe176 and Arg178.

In one embodiment the growth hormone variant has at least one point mutation in site 2 (N-term and Helix 1 (1-35) and/or Loop 2/Helix 3 (99-127)).

In one embodiment the growth hormone variant has at least one point mutation in N-term and Helix 1 such in positions corresponding to Phe1, Pro2, Ile4, Pro5, Leu6, Arg8, Leu9, Asn12, Ala13, Leu15, Arg16 and Arg19.

In one embodiment the growth hormone variant has at least one point mutation in Loop2/Helix3 such as in positions corresponding to Val102, Tyr103, Asn109, Asp116, Leu117, Glu119, Gly120 and Thr123.

In one embodiment the growth hormone variant has at least one point mutation in Helix 3 (AA107-127), such as in positions corresponding to Asn109, Asp116, Leu117, Glu119, Gly120 and Thr123.

In one embodiment the growth hormone variant has a point mutation in the position corresponding to G120.

In one embodiment the growth hormone variant has at least one point mutation in the positions corresponding to Lys41, Arg64, Lys172 and/or Gly120.

In one embodiment the growth hormone variant has at least two point mutations in the positions corresponding to Lys41, Arg64, Lys172 and/or Gly120.

In one embodiment the growth hormone variant has at least three point mutations in the positions corresponding to Lys41, Arg64, Lys172 and/or Gly120.

In one embodiment the growth hormone variant has at least one point mutation in the positions corresponding to Lys41, Arg64 and/or Lys172.

In one embodiment the growth hormone variant has at least two point mutations in the positions corresponding to Lys41, Arg64 and/or Lys172.

In one embodiment the growth hormone variant has exactly one, two or three point mutation(s). In one embodiment the growth hormone variant has single mutation in a position corresponding to Lys41, Arg64 or Lys172.

In one embodiment the growth hormone variant has one point mutation selected from the group of K41A, R64A and K172A. In one embodiment the growth hormone variant has two point mutations selected from the group of K41A, R64A and K172A. In one embodiment the growth hormone variant has the point mutation K41A. In one embodiment the growth hormone variant has the point mutation R64A. In one embodiment the growth hormone variant has the point mutation K172A.

In one embodiment the growth hormone variant has the point mutations K41A and R64A. In one embodiment the growth hormone variant has the point mutations R64A and K172A. In one embodiment the growth hormone variant has the point mutations K41A and K172A. In one embodiment the growth hormone variant has the three point mutations K41A, R64A and K172A.

In further embodiments the growth hormone variant has one or more deletions of one or more amino acid residues of the above mentions regions, such as deletion of at least one amino acid residue in site 1 or site 2. In one such embodiment the growth hormone variant has deletion(s) of at least one, two or three amino acid residues in the positions corresponding to Lys41, Arg64, Lys172 and/or Gly120.

Point mutations may be substitution of one or more amino acid residues, as mentioned above, by an alanine (A) residue.

In such embodiments, one or more of Lys41, Arg64 and Lys72, may be substituted by an alanine (A) residue, while Gly120 may be substituted by and arginine (R).

### Stability

A general concern for the development of alternative growth hormone compounds is the stability of such compounds. Stability may be relevant in various situations, such as during production, during storage, in use and in the patient. In the evaluation of new compounds it may be use full to select compound having resistance to certain proteases or compounds that has an extended in-vivo half-life. An increased stability is considered advantageous for both the growth hormone variant separately as well as for the compound comprising a growth hormone variant linked to an immunoglobulin Fc region.

In one embodiment a growth hormone compound comprises a growth hormone variant that is stabilized towards proteolytic degradation. Non-limiting examples of growth hormone proteins that are stabilized towards proteolytic degradation (by specific mutations) may be found in WO 2011/089250.

Protease-stabilized growth hormone variants include variants where an additional di-sulfide bond is introduced. The additional di-sulfide bridge preferably connects L3 with Helix 2. The growth hormone variant may thus according to the invention preferably comprise a pair of mutations corresponding to L73C/S132C, L73C/F139C, R77C/1138C, R77C/F139C, L81C/Q141C, L81C/Y143C, Q84C/Y143C, Q84C/S144C, S85C/Y143C, S85C/S144C, P89C/F146C, F92C/F146C or F92C/T148C in SEQ ID No.1. In a further embodiment the growth hormone variant comprises a pair of mutations corresponding to L81C/Y143C, Q84C/Y143C, S85C/Y143C, S85C/S144C or F92C/T148C in SEQ ID No. 1. Introduction of two extra cysteine residues in preferred embodiments substitutes the wild type amino acid residues in positions corresponding to AA84 or AA85 in H2 and AA143 or AA144 in L3 of SEQ ID No. 1.

Growth hormone variants according to the invention may in further embodiments include both stabilizing mutations and receptor interaction mutations described above. In a preferred embodiment a stabilizing di-sulfide is included in combination with a mutation in Site 1, such as Lys41, Arg64 and/or Lys172. Examples of such variants are variants with a mutation pair selected from: Q84C/Y143C, Q84C/S144C, S85C/Y143C and S85C/S144C which in addition includes one or more mutations in a position corresponding to Lys41, Arg64 and/or Lys172, where of the latter, as described above, is preferable substituted by an Ala residue.

### Growth Hormone “Activity”

As described herein it has surprisingly be found that growth hormone compounds comprising a growth hormone variant and an immunoglobulin Fc region has an unexpected prolonged half-life and furthermore that such compounds may have unexpected advantageous functionalities. The growth hormone variants of the compounds of the invention are unusual in character as the mutation(s)/deletion(s)/addition of one or more amino acid residues at least partially alter the functionality of the variant compared to wild type human growth hormone. The altered functionality may also be measured at the compound level e.g. by comparing the GH-variant-Fc molecules with hGH-Fc molecule.

The functionality of human growth hormone and variants thereof can be measured at multiple levels, such as the ability of the protein to interact with the growth hormone receptor (GHR) by measuring binding affinity in a surface plasma resonance binding (SPR) assay. The receptor component may be the full length human GHR or a partial receptor which includes one of more receptor regions, in particularly the extracellular domain which is responsible for hGH interaction. As described in method B herein, the extracellular domain or hGHBP is frequently used.

In one embodiment the growth hormone variant or growth hormone compound according to the invention has an affinity to GHR which is decreased compared to hGH or the equivalent compound comprising the hGH sequence (SEQ ID NO 1).

In further embodiments the invention relates to a growth hormone compound, wherein the growth hormone variant has reduced receptor affinity. In one such embodiment the growth hormone variant has a lower binding affinity to hGHR compared to human growth hormone identified by SEQ ID NO 1. In one embodiment the growth hormone variant has a lower binding affinity to hGHR via site 1 compared to human growth hormone identified by SEQ ID NO 1. The binding affinity to hGHR may be measured by any suitable assay known in the art such as an SPR assay and optionally using a biacore system.

The binding affinity between two molecules through a monovalent interaction may be quantified by determining the equilibrium dissociation constant (K_{D}). In turn, K_{D} can be determined by measurement of the kinetics of complex formation and dissociation, e.g. by the SPR method. The rate constants corresponding to the association and the dissociation of a monovalent complex are referred to as the association rate constant kₐ (or kₒₙ) and dissociation rate constant k_{d} (or k_{off}), respectively. K_{D} is related to kₐ and k_{d} through the equation K_{D}=k_{d}/kₐ. Following the above definition, binding affinities associated with different molecular interactions, such as comparison of the binding affinity of different ligands for a given receptor, may be compared by comparison of the K_{D} values for the individual complexes.

The value of the equilibrium dissociation constant can also be determined directly by well-known methods. Standard assays to evaluate the binding ability of ligands are known in the art and include, for example, ELISAs, Western blots, RIAs, and flow cytometry analysis. The binding kinetics and binding affinity can be assessed by standard assays known in the art, such as SPR assay. The affinity for a GH variant may thus be is measured by calculation of the equilibrium dissociation constant K_{D}, e.g. the ratio of concentration of free reagents and complex at equilibrium (K_{D}=[A]*[B]/[AB]). A low K_{D} indicates a stronger binding and K_{D}'s in the nano molar range are generally considered of high affinity. The K_{D} of hGH to hGHR is around 0.9 nmol.

In one embodiment the affinity (K_{D}) of a GH variant to hGHR is less than 0.9 nmol, such as 1.5 nmol.

In one embodiment the K_{D} of a GH variant to hGHR is more than 1.0 nmol, such as more than 5 nmol such as more than 50 nmol, such as more than 100 nmol, such as more than 200 nmol, such as more than 500 nmol.

In on embodiment the growth hormone variant has a lower binding affinity to hGHR via site 1 compared to human growth hormone identified by SEQ ID NO 1, wherein said binding affinity to hGHR via site 1 is measured by SPR (biacore) and the ratio of SPR K_{D} of hGH to the GH variant is less than 1, such as less than 0.5, such as 0.05, such as 0.005 or such as 0.0005.

The affinity of a growth hormone compound according to the invention may be lower than the affinity of the equivalent compound including the wild type hGH sequence.

In one embodiment the growth hormone compound has a lower binding affinity to hGHR via site 1 compared to a compound where human growth hormone identified by SEQ ID NO 1 is linked to an antibody Fc-domain (hGH-Fc).

In one embodiment the growth hormone compound has a lower binding affinity to hGHR via site 1 of hGH compared to a compound where human growth hormone identified by SEQ ID NO 1 is linked to an antibody Fc-domain (hGH-Fc), wherein said binding affinity to hGHR via site 1 of hGH is measured in an SPR assay and optionally using a biacore system.

A hGH-Fc (IgG) compound has an K_{D} of about 0.43 nmol. In one embodiment the growth hormone compound according to the invention binds hGHR via site 1 with an affinity (K_{D}) above 0.5 nmol, such as more than 1.0 nmol, such as more than 5 nmol such as more than 50 nmol, such as more than 100 nmol, such as more than 200 nmol, such as more than 500 nmol.

In one embodiment the growth hormone compound according to the invention binds hGHR via site 1 with an affinity (K_{D}) below 5000 nmol, such as less than 4000 nmol, such as less than 2500 nmol such as less than 1000 nmol, such as less than 750 nmol, such as less than 500 nmol.

In one embodiment the growth hormone compound according to the invention binds hGHR via site 1 with an affinity (K_{D}) between 5000-0.5 nmol, such as between 4000-1.0 nmol, between 2500-10 nmol, between 1000-25 nmol, between 500-50 nmol, between 250-75 nmol.

In one embodiment the growth hormone compound has a lower binding affinity to hGHR via site 1 compared to a compound where human growth hormone identified by SEQ ID NO 1 is linked to said antibody Fc-domain (wt GH-Fc), wherein said binding affinity to hGHR via site 1 is measured by SPR (biacore) and the ratio of SPR K_{D} of hGH-Fc to the GH variant-Fc is less than 1, such as less than 0.5, such as 0.05, such as 0.005 or such as 0.0005.

As described here in above the present invention relates to the finding that variants with functionalities different from the wild type sequence may have advantageous characteristics in combination with an Fc-domain. Usually mutations are introduced to increase specific functionalities but the present invention relates to the opposite situation were mutations in hGH are introduced to decrease affinity to the receptor and/or signalling through the receptor.

Although binding to the receptor is of high importance an alternative measure is in vitro activity. The binding of a growth hormone variant or a GH-variant-Fc compound to stimulate signalling through the GHR receptor may be measured in an in vitro cell based assay such as a BAF assay (method C herein).

In one embodiment the in vitro activity of the hGH variant or the GH-variant-Fc compound is lower compared to hGH or the equivalent Fc compounds comprising the hGH sequence (SEQ ID NO 1).

In one embodiment the growth hormone variant has a lower in vitro activity compared to human growth hormone identified by SEQ ID NO 1. In one embodiment the growth hormone compound has a lower in vitro activity compared to a compound where hGH (SEQ ID NO 1) is linked to an antibody Fc-domain. In one such embodiment the in vitro activity is measured in a BAF assay. In one embodiment the growth hormone variant has a lower in vitro activity in a BAF assay compared to human growth hormone identified by SEQ ID NO 1. In one embodiment the growth hormone variant has a lower in vitro activity in a BAF assay compared to human growth hormone identified by SEQ ID NO 1, wherein the ratio of GH variant to hGH is more than 1, such as more than 2, such as more than 5.

In one embodiment the growth hormone variant has a lower in vitro activity in a BAF assay compared to human growth hormone identified by SEQ ID NO 1, wherein the ratio of the GH variant to wt hGH is 1-200, such as 1-100, such as 1-50, such as 1-25, such as 1-15, such as 1-10.

In one embodiment the growth hormone variant has a lower in vitro activity in a BAF assay compared to human growth hormone identified by SEQ ID NO 1, wherein the ratio of the GH variant to wt hGH is 2-25, such as 2-15, such as 2-10.

In one embodiment the growth hormone compound has a lower in vitro activity in a BAF assay compared to a compound where hGH identified by SEQ ID NO 1 is linked to an antibody Fc-domain.

In one embodiment the growth hormone compound has a lower in vitro activity in a BAF assay compared to a compound where human growth hormone identified by SEQ ID NO 1 is linked to an antibody Fc-domain (hGH-Fc), wherein the BAF ratio of said GH variant-Fc to hGH-Fc is more than 1, such as more than 2, such as more than 5, such as more than 10, such as more than 25, such as more than 50.

In one embodiment the growth hormone compound has a lower in vitro activity in a BAF assay compared to a compound where hGH (SEQ ID NO 1) is linked to an antibody Fc-domain (hGH-Fc), wherein the BAF ratio of said GH variant-Fc to hGH-Fc is 1-200, such as 1-100, such as 1-50, such as 1-25, such as 1-15, such as 1-10.

In one embodiment the growth hormone compound has a lower in vitro activity in a BAF assay compared to a compound where hGH (SEQ ID NO 1) is linked to said antibody Fc-domain (wt GH-Fc), wherein the BAF ratio of said GH variant-Fc to wt GH-Fc is 2-25, such as 2-15, such as 2-10.

So far, as the purpose of creating new growth hormone compounds is the aim of identifying new therapeutic compounds that has the potential to substitute and preferably improve the treatment options for patients that benefit from a higher level of circulating growth hormone, it is of significant relevance to confirm that the a given compound has the ability to stimulate signalling of the GH receptor.

### Half-Life (T_{1/2})

The half-life (T_{1/2}) of a growth hormone variant or compound is the time required for reducing the quantity by one half.

The growth hormone variant or compound preferably has an increased T_{1/2} compared to wild type human growth hormone or an equivalent compound including the hGH sequence, which may be provided by various means known to the person skilled in the art, such as point mutations stabilizing the protein from degradation. The circulation time of a growth hormone compound may also be increased by covalent or non-covalent linkage to serum proteins. Serum albumin may be used by conjugation (optionally including a linker) or by protein fusion with a growth hormone or variant thereof. Alternatively chemical linkage to albumin may also be considered as well as fusion or linkage with antibody Fc regions. Non-covalent attachment to albumin may be obtained through the use of albumin binders such as acyl groups (albumin binders) covalently bond to growth hormone. Alternative protracting moieties may be used as known in the art.

Rat models are frequently used to test biological effect of growth hormone variants and compounds. Testing may be performed in normal rats and/or in hypophysectomised rats. The Sprague Dawley rat is frequently used and methods for testing are described in Method D and E. Such testing may provide info on several pharmacokinetic parameters such as the AUC,T½ and MRT (mean residence time) which are relevant to determine the total exposure and the duration of the presence of a given compound in the blood of a recipient. Furthermore an induction of the IGF-1 response, characteristic for the biological effect of hGH, can be measured.

In one embodiment the growth hormone variant has an increased half-life compared to hGH (SEQ ID NO 1).

In one embodiment the growth hormone compound according to the invention has an increased in vivo T_{1/2} compared to hGH (SEQ ID NO 1).

It is noted that human growth hormone (hGH) has a T_{1/2} of approximately 12-14 minutes in the described assay (method D herein). Although not equivalent with half-life in humans, it is contemplated that an increased in vivo T_{1/2} in rats will also translate into an extended in vivo presence in a therapeutic setting.

In one embodiment the growth hormone compound has a T_{1/2} above 30 minutes, or above 60 minutes, or above 90 minutes or above 120 minutes. In further embodiments T_{1/2} is above 60 minutes or 1 hour, such as above 2 hours or preferably above 4 hours.

As mentioned above it may as well be relevant to consider the in vivo half-life of either the growth hormone compound or the individual growth hormone variant.

In one embodiment the growth hormone variant has an increased T_{1/2}, such as a T_{1/2} of above 30 minutes, or above 60 minutes, or above 90 minutes or above 120 minutes. In further embodiments T_{1/2} of the growth hormone variant is above 60 minutes or 1 hour, such as above 2 hours or preferably above 4 hours.

In one embodiment the extended T_{1/2} is measure is measured after intravenous (iv.) or subcutaneous (sc.) administration to rats as described in the examples. The skilled person will know how such assay can be modified, depending on the tools available for detection of the growth hormone variant or growth hormone compound.

In one embodiment the growth hormone compound has an increased half-life compared to the equivalent hGH-Fc compound. In one embodiment the growth hormone compound has a half-life (T½) of more than 8 hours, such as more than 12 hours, such as more than 24 hours. In one embodiment the growth hormone compound has a half-life (T½) of more than 8 hours, such as more than 12 hours, such as more than 24 hours, when measured after a single iv. dose of 15 nmol to normal rats.

In one embodiment the growth hormone compound has a half-life (T½) of more than 8 hours, such as more than 12 hours, such as more than 24 hours, when measured after a single iv. dose of 15 nmol to hypophysectomised rats (see method E herein).

In one embodiment the growth hormone compound has a half-life (T½) of more than 48 hours, such as more than 60 hours, such as more than 72 hours, when measured after a single iv. dose of 15 nmol to hypophysectomised rats.

In one embodiment the growth hormone variant has an increased MRT compared to hGH (SEQ ID NO 1).

In one embodiment the growth hormone compound has an increased MRT compared to the equivalent hGH-Fc compound.

In one embodiment the growth hormone compound has a MRT of more than 12 hours, such as more than 18 hours, such as more than 24 hours. In one embodiment the growth hormone compound has a MRT of more than 12 hours, such as more than 18 hours, such as more than 24 hours, when measured after a single dose iv dose of 15 nmol to normal rats.

In one embodiment the growth hormone compound has a MRT of more than 12 hours, such as more than 18 hours, such as more than 24 hours, such as more than 36 hours, such as more than 48 hours, when measured after a single dose iv dose of 15 nmol to hypophysectomised rats.

The IGF-1 response may be measured after dosing of a growth hormone compound such as described in Method F, herein, although the skilled person will know to apply alternative methods as well. The plasma concentration of IGF-1 in rats after a single dose a growth hormone should preferably increase over a period of time corresponding to the increased plasma concentration of the growth hormone compound.

In one embodiment the growth hormone variant or growth hormone compound according to the invention is capable of inducing an IGF-1 response.

An IGF-1 response may thus be stronger than the response observed for a wt compound by reaching a higher concentration of plasma IGF-1. The concentration of plasma IGF-1 may be detected within 72 hours, such as within 48 hours, such as within 36 hours, such as within 24 hours. To compare effects of different compound values may be measured at different time points and compared at each individual time point, such as by either of 6, 12, 24, 36, 48, 72, 96, 144, 192, 240, 288, 336 hours after a dosage.

In one embodiment the growth hormone variant or growth hormone compound induces an increased IGF-1 response. In one embodiment the growth hormone variant/compound induces an IGF-1 response, wherein the IGF-1 response is detected as an increased plasma IGF-1 concentration at up to 96 hours, or such as 6, 12, 24, 36, 48, 72 hour after a single dose of said growth hormone variant or compound. In one embodiment the growth hormone variant or growth hormone compound induces an extended IGF-1 response. If the plasma concentration of IGF-1 remains high over an extended period of time compared to a wt compound, the growth hormone variant/compound induces an extended an IGF-1 response. In one embodiment the growth hormone variant or growth hormone compound induces an extended IGF-1 response compared to the IGF response of wt hGH or an equivalent compound including wt hGH. In one embodiment the IGF response lasts more than 24 hours, such as more than 48 hours, such as more than 96 hours or even more than 144 hours.

An extended IGF-1 response may be detected as the area under the curve (AUC) of the plasma IGF-1 concentration of a fixed period, such as over a period of up to 336 hours, such for 6, 12, 24, 36, 48, 72, 96, 144, 192, 240, 288 or 336 hours after a dosage.

In one embodiment the growth hormone compound induces an IGF-1 response, wherein the IGF-1 response is detected after a single dose. In one embodiment the growth hormone variant/compound induces an IGF-1 response, wherein the IGF-1 response is detected after a single i.v. dose of 15 nmol.

In one embodiment the growth hormone compound induces an IGF-1 response, wherein the IGF-1 response is detected as the AUC after a single dose i.v. dose of 15 nmol.

In one embodiment the growth hormone compound induces an IGF-1 response, wherein the IGF-1 response is detected as the AUC of plasma IGF-1 up to 336 hours, such as 6, 12, 24, 36, 48, 72, 96, 144, 192, 240, 288 or 336 hours after a single i.v. dose of 15 nmol.

In one embodiment the growth hormone compound is capable of inducing an IGF-1 response, wherein said response is measured in rats.

In one embodiment the growth hormone compound is capable of inducing an IGF-1 response in normal rats. In one embodiment the growth hormone compound is capable of inducing an IGF-1 response in hypophysectomised rats.

According to the invention the IGF-1 response may be detected based on an IGF-la concentration profile or based on a baseline corrected IGF-1 concentration profile. The concentration of IGF-1 in a blood or plasma sample is measured over time after administration of the growth hormone compound and the increase above base line represent the induced IGF-1 response.

The IGF-1 response may also be described as the area under the curve (AUC) for IGF-1 in response to a single i.v. dosage.

In hypophysectomised rats the ability of a compound to induce weight gain can be measured. In one embodiment the growth hormone compound induce weight gain. In one embodiment the growth hormone compound induce weight gain in hypophysectomised rats.

In one embodiment the growth hormone compound induce weight gain in hypophysectomised rats

### Fc-Domain

The fragment crystallizable region (Fc region or Fc-domain) of an antibody is the tail of an antibody. For IgG, IgA and IgD antibodies the Fc region contains two identical protein fragments both comprising the second and third constant domains (CH2 and CH3). The Fc regions of IgM and IgE antibodies contain three heavy chain constant domains (CH domains 2-4) in each polypeptide chain. The proteins of the Fc-domain are herein referred to as Fc polypeptides and usually comprise at least the CH2 and CH3 domains.

The Fc-domain mediates interaction with cell surface receptors called Fc receptors, as well as some proteins of the complement system. The Fc region enables antibodies to interact with the immune system. The Fc region of an antibody is at least partly responsible for the long half-life of antibody molecules, which for an IgG is approximately 720 hours in humans. The Fc-domain is thus an attractive protractor for extending the half-life of potential therapeutic compounds.

According to the present invention it has been found that the use of an Fc-domain as an protractor of a growth hormone variant results in a growth hormone compounds with attractive functionalities.

In one embodiment the growth hormone compound comprises a growth hormone variant linked to an antibody Fc-domain (GH-variant-Fc), wherein the isotype of the Fc-domain is IgG, such as subtype IgG1, such as IgG2, such as IgG4.

In one embodiment the growth hormone compound comprises the CH2 and CH3 domains of human IgG1. In one embodiment the growth hormone compound comprises two Fc polypeptides each defined by SEQ ID NO 3 (11-227).

The hinge region is the protein segment between CH1 and CH2 of the constant region of the antibody. In one embodiment the Fc-polypeptide comprises a hinge region including one or more cysteines. In one embodiment the polypeptides of the Fc domain each comprises the sequence as defined by SEQ ID NO: 3. In one embodiment, the hinge region is modified such that the number of cysteine residues in the hinge region is altered, e.g., increased or decreased.

In one embodiment the constant region may be modified to stabilize the molecule, for example, in an IgG4 constant region, residue S228 (residue numbering according to the EU index) may be mutated to a proline (P). In one embodiment the Fc polypeptides includes a proline residue in position S228, or in a position corresponding to S228 in IgG4.

The Fc polypeptides may thus be covalently linked or alternatively non-covalently linked.

In one embodiment the Fc region may be engineered to include modifications within the Fc region, typically to alter one or more of its functional properties, such as serum half-life, complement fixation, Fc-receptor binding, protein stability and/or antigen-dependent cellular cytotoxicity, or lack thereof, among others.

Furthermore, an Fc-domain of the invention may be chemically modified (e.g., one or more chemical moieties can be attached to the Fc part) to alter its glycosylation, again to alter one or more functional properties of the antibody.

An IgG1 Fc-domain may comprises one or more, and perhaps all of the following mutations that will result in decreased affinity to certain Fc receptors (L234A, L235E, and G237A) and in reduced C1q-mediated complement fixation (A330S and P331S), respectively (residue numbering according to the EU index).

In order to improve binding affinity to FcRn mutations in the Fc may be included such as mutation M428L and/or N434S in an Fc-domain of the IgG1 isotype.

### Linkage

Linkage of a growth hormone variant to an Fc-domain may be obtained by multiple routes know in the art. In one embodiment the growth hormone compound comprises a growth hormone variant covalently linked to an antibody Fc-domain as described herein above.

Traditionally, creation of fusion proteins has been used to combine to protein domains. By fusing the coding region for a growth hormone variant and a Fc polypeptide a growth hormone variant Fc fusion protein may be obtained, wherein the growth hormone variant is attached to both Fc polypeptides via at least one peptide bond resulting in a growth hormone compound which is bivalent with regards to the GH component as two Fc polypeptides join to form an Fc-domain.

In one embodiment a growth hormone variant and a Fc polypeptide is linked by at least one peptide bond. In one embodiment the fusion is through a peptide linker, such as a GS linker.

In one embodiment the growth hormone compound is a bivalent fusion protein compound.

In one embodiment the growth hormone compound comprises at least one GH variant and Fc polypeptide (GH-variant-Fc polypeptide) fusion protein, where the GH variant and Fc polypeptide are linked by a linker peptide, such as a GS linker.

In an alternative embodiment only one of the two Fc polypeptides are fused with the growth hormone variant, resulting in compound that is monovalent with regards to the GH component.

In one embodiment the growth hormone compound comprises only one GH variant and one Fc-domain. The growth hormone compound may comprise one GH variant polypeptide and two Fc polypeptides. The growth hormone compound may thus comprise one GH variant polypeptide and one Fc-domain

In one embodiment the one GH variant is linked to the Fc-domain via the N-terminal or the C-terminal of said GH variant.

In further embodiments the growth hormone compound comprises only one GH variant which is linked to the Fc-domain at the N-terminal or the C-terminal of one of the Fc polypeptides.

In one embodiment the C-terminal of the GH variant is linked to the N-terminal of a Fc polypeptide.

In one embodiment the C-terminal of a Fc polypeptide is linked to the N-terminal of the GH variant.

As an alternative to the generation of fusion proteins, covalent linkage of a growth hormone variant and an Fc-domain may be obtained by conjugation. Methods for such process are known in the art which will involve non-peptide bonds, and examples of conjugation may be found in the references provided in the background section.

As evident from the disclosure herein the present invention in an aspect relates to method for preparing a growth hormone compound with increased plasma half-life comprising including a growth hormone variant as has been defined herein above. The use of such variant compared to use of the human growth hormone sequence (SEQ ID NO. 1) in itself provides an increase in vivo half-life independent of any protracting moiety of a given compound. In particular embodiments hereof the growth hormone variant is used in combination with an Fc-domain.

### Methods of Preparation

Generation of growth hormone compounds may be performed by various methods known in the art. Standard methods involve cloning of the protein components and subsequence expression in a suitable host.

The growth hormone variant may be produced by means of recombinant nucleic acid techniques. In general, a cloned wild-type growth hormone nucleic acid sequence is modified to encode the desired variant. This modified sequence is then inserted into an expression vector, which is in turn transformed or transfected into host cells.

The nucleic acid construct encoding the growth hormone may suitably be of genomic, cDNA or synthetic origin. Amino acid sequence alterations are accomplished by modification of the coding region by well-known techniques, such as by using quick change site-directed mutagenesis kit's. One embodiment relates to a nucleotide sequence or construct encoding a growth hormone variant according to the present invention.

In one embodiment the invention relate to an isolated nucleotide sequence or construct encoding a GH variant and Fc polypeptide fusion according to the invention.

The DNA sequences encoding the growth hormone variants are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the growth hormone variant is operably linked to additional segments required for transcription of the DNA. The term, “operably linked” indicates that the segments are arranged so that they function in concert for their intended purposes, e.g. transcription initiates in a promoter and proceeds through the DNA sequence coding for the polypeptide until it terminates within a terminator.

Thus, expression vectors for use in expressing growth hormone variants will comprise a promoter capable of initiating and directing the transcription of a cloned gene or cDNA. The promoter may be any DNA sequence, which shows transcriptional activity in the host cell of choice and may be derived from genes encoding proteins either homologous or heterologous to the host cell.

Additionally, expression vectors for use of expression of growth hormone variants will also comprise a terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

Expression of growth hormone variants can aim for either intracellular expression in the cytosol of the host cell or be directed into the secretory pathway for extracellular expression into the growth medium.

Intracellular expression is the default pathway and requires an expression vector with a DNA sequence comprising a promoter followed by the DNA sequence encoding the growth hormone variants polypeptide followed by a terminator.

To direct the growth hormone variants into the secretory pathway of the host cells, a secretory signal sequence (also known as signal peptide or a pre sequence) is needed as an N-terminal extension of the growth hormone variants. A DNA sequence encoding the signal peptide is joined to the 5′ end of the DNA sequence encoding the growth hormone variants in the correct reading frame. The signal peptide may be that normally associated with the protein or may be from a gene encoding another secreted protein. For growth hormone variant a cleavable N-terminal tag is frequently used to ensure an N-terminal identical to the mature human growth hormone can be obtained. The DNA sequence coding for the variant is linked in-frame with codons encoding a small peptides such as MEAE which are cleaved of during the further processing. For some purposes a single Met-start codon can be added to allow translation start. The tag may additionally serve as a purification tag in the further processing of the protein. A method of expressing a growth hormone compound according to the invention is described in the Examples (Method A), and the skilled person will understand how to modify the method to obtain alternative compounds.

In embodiments were linkage of the growth hormone variant to the Fc-domain is obtained using peptide bonds the most convenient route to obtain such compounds is by creation of a single nucleotide sequence encoding the growth hormone variant and the Fc polypeptide in-frame. The sequence may further encode a linking peptide. The order may be in either of the two possibilities e.g. the GH-variant may be N-terminal to the Fc polypeptide or the GH-variant may be C-terminal to the Fc polypeptide.

The procedures used to ligate the DNA sequences coding for the GH-variant, the Fc polypeptide, the linking peptide, the promoter, the terminator, purification tag and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N. Y., 1989).

In one embodiment the invention relates to an expression vector encoding a GH variant or a GH variant and a Fc polypeptide fusion optionally including a linking peptide.

The host cell into which the DNA sequences encoding growth hormone variant is introduced may be any cell that is capable of expressing growth hormone variant either intracellular or extracellular. If posttranslational modifications are needed, suitable host cells include yeast, fungi, insects and higher eukaryotic cells such as mammalian cells including HEC cells as described in Method A herein. Host cells expressing a GH variant or a GH variant—Fc polypeptide fusion are further embodiments of the invention.

As described herein above, growth hormone compounds of the invention may be either monovalent or bivalent with regards to the growth hormone variant. Expression of bivalent growth hormone variant—Fc molecules can be obtained by expression of a single nucleotide sequence encoding both proteins. Expression of monovalent growth hormone variant—Fc molecules requires further considerations as a separate Fc polypeptide must by expressed and a heterologous complex of a growth hormone variant—Fc-polypeptide and the second Fc polypeptide must be obtained.

Depending on the sequence of the Fc polypeptide the complex may be covalently or non-covalently formed. The Fc polypeptide may include cysteine residues capable of forming disulfide bonds, which are usually present in the hinge region of an immunoglobulin molecule and may thus be included in the Fc polypeptide.

Complex formation of the GH-variant—Fc-polypeptide and the second Fc polypeptide may be obtained by expressing both coding frames in the same cell or by subsequent mixing of polypeptides if expressed in individual host cell cultures. The first method is preferably and may be followed by purification of the protein complex constituting the growth hormone compound e.g. a GH-variant-Fc.

The purification of polypeptides or protein complexes may be performed using standard procedures known in the art.

The invention further relates to a method for preparing a growth hormone compound according to the invention comprising the steps of:
a) obtaining a host cell expressing a growth hormone variant polypeptide,
b) obtaining a host cell expressing an Fc polypeptide capable of forming an Fc-domain by association of two Fc-polypeptides,
c) expressing and purifying said growth hormone variant polypeptide and said Fc-domain formed by two Fc-polypeptides from said host cell(s),
d) linking the growth hormone variant polypeptide and the Fc-domain and
e) obtaining said growth hormone compound.

The invention further relates to a method for preparing a growth hormone compound according to the invention, wherein the compound comprises a GH-variant-Fc polypeptide fusion, comprising the steps of:
a) obtaining a host cell (1) expressing a GH-variant-Fc polypeptide fusion,
b) optionally obtaining a host cell (2) expressing a Fc polypeptide, wherein said host cell (2) may be identical to host cell (1) of a),
c) expressing and purifying said polypeptide(s) from said host cell(s) and
d) obtaining a growth hormone compound said compound comprising a GH-variant-Fc polypeptide fusion.

In a further embodiment the compound is a GH-variant-Fc domain. In a further embodiment the host cell (1) and (2) are identical.

### Pharmaceutical Compositions

Accordingly, one object of the invention is to provide a pharmaceutical formulation comprising such a growth hormone compound in a therapeutically active amount. As described above the concentration may vary from 0.25 mg/ml to 250 mg/ml in a solution or 2.5 mg/g to 250 mg/g in a solid dosage form. It is preferred that said formulation has a pH from 2.0 to 10.0. The formulation may further comprise a buffer system, a preservative, a tonicity agent, a chelating agent, a stabilizer, and/or a surfactant, as well as various combinations thereof. The use of preservatives, isotonic agents, chelating agents, stabilizers and surfactants in pharmaceutical compositions is well-known to the skilled person. The formulations may be prepared using standard procedures know in the art. Reference may be made to Remington: The Science and Practice of Pharmacy, 19th edition, 1995. In one embodiment the pharmaceutical composition according to the present invention is a liquid.

### Method of Treatment

An aspect of the invention relates to the use of growth hormone compound and composition hereof in methods of treatment. In one embodiment the growth hormone compound and composition hereof is for use in method of treatment.

In one embodiment, the growth hormone compound and compositions hereof according is used for the preparation of a medicament.

In such embodiments, the growth hormone compound or the pharmaceutical composition according to the invention is for treatment or prevention of growth hormone deficiency in children and adults. Other diseases or disorders where an increased concentration of circulating growth hormone may be helpful may also be treated or prevented using the growth hormone compound or pharmaceutical composition of the invention. In one embodiment the growth hormone compound or pharmaceutical compositions of the invention is for treating diseases or states where a benefit from an increase in the amount of circulating growth hormone is observed. Such diseases or states include growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Chron's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children. Growth hormones have also been used for acceleration of the healing of muscle tissue, nervous tissue or wounds; the acceleration or improvement of blood flow to damaged tissue; or the decrease of infection rate in damaged tissue.

In one embodiment, the growth hormone compound and compositions hereof is for treatment of GHD in children, GHD in adults (AGHD), Turner syndrome (TS), Noonan syndrome, Idiopathic short stature (ISS), Small for gestational age (SGA), Prader-Willi syndrome (PWS), Chronic renal insufficiency (CRI), Skeletal dysplasia, SHOX deficiency AIDS wasting, HIV associated lipdystrophy (HARS), Short bowel syndrome optionally including, steroid dependent disease, cystic fibrosis and fibromyalgia.

In one embodiment, the present invention relates to a method of treating diseases or states mentioned above, wherein the activity of the growth hormone compound is useful for treating said diseases or states. The administering of such compounds resulting in a therapeutic benefit associated with an increase in the amount of circulating growth hormone compound in the patient. In an embodiment said method comprises, administering to a patient an effective amount of a pharmaceutical composition of a growth hormone compound thereby ameliorating the symptoms of said patient.

In one embodiment, the present invention relates to a method comprising administration to a patient in need thereof an effective amount of a therapeutically effective amount of a pharmaceutical composition according to the invention comprising a growth hormone compound. The present invention thus provides a method for treating these diseases or states, the method comprising administering to a patient in need thereof a therapeutically effective amount of a growth hormone compound in a pharmaceutical composition according to the present invention.

A “therapeutically effective amount” of a compound of the invention as used herein means an amount sufficient to cure, alleviate or partially arrest the clinical manifestations of a given disease and its complications. An amount adequate to accomplish this is defined as “therapeutically effective amount”. Effective amounts for each purpose will depend on e.g. the severity of the disease or injury as well as the weight, sex, age and general state of the subject. It will be understood that determining an appropriate dosage may be achieved using routine experimentation, which is all within the ordinary skills of a trained physician or veterinary.

In one embodiment, the invention provides the use of a growth hormone compound or its conjugate in the manufacture of a medicament used in the treatment of the above mentioned diseases or states.

The present invention in further aspects relates to methods involving the pharmaceutical composition described herein above. The invention is further described by the following non-limiting embodiments.

### EMBODIMENTS

1. A growth hormone compound comprising a growth hormone variant linked to an antibody Fc-domain (GH-variant-Fc).
2. The growth hormone compound, according to embodiment 1, wherein the growth hormone variant has one or more amino acid substitution(s), deletion(s) and/or addition(s) compared to hGH (SEQ ID NO 1).
3. The growth hormone compound, according to embodiment 1-2, wherein the growth hormone variant has at most 5 amino acid substitution(s), deletion(s) and/or addition(s) compared to hGH (SEQ ID NO 1).
4. The growth hormone compound, according to any of the previous embodiments, wherein the growth hormone variant has improved stability.
5. The growth hormone compound, according to any of the previous embodiments, wherein the growth hormone variant has improved stability compared to human growth hormone identified by SEQ ID NO 1.
6. The growth hormone compound, according to any of the previous embodiments, wherein the growth hormone variant has improved protease resistance compared to human growth hormone identified by SEQ ID NO 1.
7. The growth hormone compound, according to any of the previous embodiments, wherein the growth hormone variant has reduced receptor affinity.
8. The growth hormone compound, according to any of the previous embodiments, wherein the growth hormone variant has a lower binding affinity to GHR compared to human growth hormone identified by SEQ ID NO 1.
9. The growth hormone compound, according to any of the previous embodiments, wherein the growth hormone variant has a lower binding affinity to hGHR via site 1 compared to human growth hormone identified by SEQ ID NO 1.
10. The growth hormone compound, according to any of the previous embodiments, wherein the growth hormone variant has a lower binding affinity to hGHR via site 1 compared to human growth hormone identified by SEQ ID NO 1, wherein said binding affinity to hGHR via site 1 is measured by SPR (biacore).
11. The growth hormone compound, according to any of the previous embodiments, wherein the growth hormone variant binds hGHR via site 1 with an affinity (KD) between 5000-0.5 nmol, such as between 4000-1.0 nmol, between 2500-10 nmol, between 1000-25 nmol, between 500-50 nmol, between 250-75 nmol.
12. The growth hormone compound, according to any of the previous embodiments, wherein the growth hormone compound has a lower binding affinity to hGHR compared to a compound where human growth hormone identified by SEQ ID NO 1 is linked to said antibody Fc-domain (wt GH-Fc).
13. The growth hormone compound, according to any of the previous embodiments, wherein the growth hormone compound has a lower binding affinity to hGHR via site 1 compared to a compound where human growth hormone identified by SEQ ID NO 1 is linked to said antibody Fc-domain (wt GH-Fc).
14. The growth hormone compound, according to any of the previous embodiments, wherein the growth hormone compound has a lower binding affinity to hGHR via site 1 compared to a compound where human growth hormone identified by SEQ ID NO 1 is linked to said antibody Fc-domain (wt GH-Fc), wherein said binding affinity to hGHR via site 1 is measured by SPR (biacore).
15. The growth hormone compound, according to any of the previous embodiments, wherein the growth hormone compound binds hGHR via site 1 with an affinity (KD) between 5000-0.5 nmol, such as between 4000-1.0 nmol, between 2500-10 nmol, between 1000-25 nmol, between 500-50 nmol, between 250-75 nmol.
16. The growth hormone compound, according to any of the previous embodiments, wherein the growth hormone variant has a lower in vitro activity compared to human growth hormone identified by SEQ ID NO 1.
17. The growth hormone compound, according to any of the previous embodiments, wherein the growth hormone compound has a lower in vitro activity compared to a compound where human growth hormone identified by SEQ ID NO 1 is linked to said antibody Fc-domain.
18. The growth hormone compound, according to any of the previous embodiments 16 and 17, wherein the in vitro activity is measured in a BAF assay.
19. The growth hormone compound, according to any of the previous embodiments, wherein the growth hormone variant has a lower in vitro activity in a BAF assay compared to human growth hormone identified by SEQ ID NO 1.
20. The growth hormone compound, according to any of the previous embodiments, wherein the growth hormone variant has a lower in vitro activity in a BAF assay compared to human growth hormone identified by SEQ ID NO 1, wherein the ratio of GH variant to wt GH is more than 1, such as more than 2, such as more than 5.
21. The growth hormone compound, according to any of the previous embodiments, wherein the growth hormone variant has a lower in vitro activity in a BAF assay compared to human growth hormone identified by SEQ ID NO 1, wherein the ratio of the GH variant to wt hGH is 1-200, such as 1-100, such as 1-50, such as 1-25, such as 1-15, such as 1-10.
22. The growth hormone compound, according to any of the previous embodiments, wherein the growth hormone variant has a lower in vitro activity in a BAF assay compared to human growth hormone identified by SEQ ID NO 1, wherein the ratio of the GH variant to wt hGH is 2-25, such as 2-15, such as 2-10.
23. The growth hormone compound, according to any of the previous embodiments, wherein the growth hormone compound has a lower in vitro activity in a BAF assay compared to a compound where human growth hormone identified by SEQ ID NO 1 is linked to said antibody Fc-domain.
24. The growth hormone compound, according to any of the previous embodiments, wherein the growth hormone compound has a lower in vitro activity in a BAF assay compared to a compound where human growth hormone identified by SEQ ID NO 1 is linked to said antibody Fc-domain (wt GH-Fc), wherein the BAF ratio of said GH variant-Fc to wt GH-Fc is more than 1, such as more than 2, such as more than 5.
25. The growth hormone compound, according to any of the previous embodiments, wherein the growth hormone compound has a lower in vitro activity in a BAF assay compared to a compound where human growth hormone identified by SEQ ID NO 1 is linked to said antibody Fc-domain (wt GH-Fc), wherein the BAF ratio of said GH variant-Fc to wt GH-Fc is 1-200, such as 1-100, such as 1-50, such as 1-25, such as 1-15, such as 1-10.
26. The growth hormone compound, according to any of the previous embodiments, wherein the growth hormone compound has a lower in vitro activity in a BAF assay compared to a compound where human growth hormone identified by SEQ ID NO 1 is linked to said antibody Fc-domain (wt GH-Fc), wherein the BAF ratio of said GH variant-Fc to wt GH-Fc is 2-25, such as 2-15, such as 2-10.
27. The growth hormone compound, according to any of the previous embodiments, wherein the growth hormone compound has an increased in vivo half-life (T½).
28. The growth hormone compound, according to any of the previous embodiments, wherein the growth hormone compound has an increase in vivo half-life compared to the equivalent hGH-Fc compound.
29. The growth hormone compound, according to any of the previous embodiments, wherein the growth hormone compound has an in vivo half-life of more than 8 hours, such as more than 12 hours, such as more than 24 hours.
30. The growth hormone compound, according to any of the previous embodiments, wherein the growth hormone compound has an in vivo half-life of more than 8 hours, such as more than 12 hours, such as more than 24 hours, when measured after a single iv dose to normal or hypophysectomised rats.
31. The growth hormone compound, according to any of the previous embodiments, wherein the growth hormone compound has an in vivo half-life of more than 8 hours, such as more than 12 hours, such as more than 24 hours, when measured after a single iv dose of 15 nmol to normal or hypophysectomised rats.
32. The growth hormone compound, according to any of the previous embodiments, wherein the growth hormone compound has an increased MRT.
33. The growth hormone compound, according to any of the previous embodiments, wherein the growth hormone compound has an increased MRT compared to the equivalent hGH-Fc compound.
34. The growth hormone compound, according to any of the previous embodiments, wherein the growth hormone compound has an increased MRT compared to the equivalent hGH-Fc compound when measured after a single iv dose to normal or hypophysectomised rats.
35. The growth hormone compound, according to any of the previous embodiments, wherein the growth hormone compound has a MRT of more than 12 hours, such as more than 18 hours, such as more than 24 hours.
36. The growth hormone compound, according to any of the previous embodiments, wherein the growth hormone compound has a MRT in vivo of more than 12 hours, such as more than 18 hours, such as more than 24 hours, when measured after a single dose iv. dose of 15 nmol to normal rats.
37. The growth hormone compound, according to any of the previous embodiments, wherein the growth hormone compound has a MRT in vivo of more than 12 hours, such as more than 18 hours, such as more than 24 hours, such as more than 36 hours, such as more than 48 hours, when measured after a single dose iv dose of 15 nmol to hypophysectomised rats.
38. The growth hormone compound, according to any of the previous embodiments, wherein the growth hormone compound is capable of inducing an IGF-1 response.
39. The growth hormone compound, according to any of the previous embodiments, wherein the growth hormone compound induces an increased IGF-1 response.
40. The growth hormone compound, according to any of the previous embodiments, wherein the growth hormone compound induces an extended IGF-1 response.
41. The growth hormone compound, according to any of the previous embodiments, wherein the growth hormone compound induces an IGF-1 response, wherein the IGF-1 response is detectable after a single iv dose.
42. The growth hormone compound, according to any of the previous embodiments, wherein the growth hormone compound induces an IGF-1 response, wherein the IGF-1 response is detected as the AUC of plasma IGF-1 concentration after a single iv dose.
43. The growth hormone compound, according to any of the previous embodiments, wherein the growth hormone compound induces an IGF-1 response, wherein the IGF-1 response is detected as an increased plasma IGF-1 concentration at up to 96 hours after a single dose iv dose.
44. The growth hormone compound, according to any of the previous embodiments, wherein the growth hormone compound induces an IGF-1 response, wherein the IGF-1 response is detected as the AUC of plasma IGF-1 concentration up to 336 hours after a single dose iv dose of 15 nmol.
45. The growth hormone compound, according to any of the previous embodiments, wherein the growth hormone compound induces an extended IGF-1 response, which lasts more than 24 hours, such as more than 48 hours, such as more than 96 hours or even more than 144 hours.
46. The growth hormone compound, according to any of the previous embodiments, wherein the growth hormone compound induces an IGF-1 response, wherein the IGF-1 response is detected based on an IGF-1 concentration profile or based on a baseline corrected IGF-1 concentration profile.
47. The growth hormone compound, according to any of the previous embodiments, wherein the growth hormone compound induces an IGF-1 response, wherein said response is detected in rats.
48. The growth hormone compound, according to any of the previous embodiments, wherein the growth hormone compound induces an IGF-1 response in normal rats.
49. The growth hormone compound, according to any of the previous embodiments, wherein the growth hormone compound induces an IGF-1 response in hypophysectomised rats.
50. The growth hormone compound, according to any of the previous embodiments, wherein the growth hormone compound induces weight gain.
51. The growth hormone compound, according to any of the previous embodiments, wherein the growth hormone compound induces weight gain in hypophysectomised rats.
52. The growth hormone compound, according to any of the previous embodiments, wherein the growth hormone variant has at least one mutation compared to human growth hormone identified by SEQ ID NO 1.
53. The growth hormone compound, according to any of the previous embodiments, wherein the growth hormone variant has at least one point mutation in site 1 (Helix 1(9-35) and Loop 1 (36-71) and Helix 4 (155-184)).
54. The growth hormone compound, according to any of the previous embodiments, wherein the growth hormone variant has at least one point mutation in Helix 1, such as a mutation in position corresponding to Met14, His18, His21, Gln22 and Phe25.
55. The growth hormone compound, according to any of the previous embodiments, wherein the growth hormone variant has at least one point mutation in Loop 1, such as a mutation in position corresponding to Lys41, Tyr42, Leu45, Gln46, Asn47, Pro48, Gln49, Ser51, Leu52, Pro59, Pro61, Ser62, Asn63, Arg64, Thr67, Glu65 and Gln68 56. The growth hormone compound, according to any of the previous embodiments, wherein the growth hormone variant has at least one point mutation in Loop 1, such as a mutation in position corresponding to Lys41, Leu45, Pro59, Pro61, Arg64 and Glu65.
57. The growth hormone compound, according to any of the previous embodiments, wherein the growth hormone variant has at least one point mutation in Helix 4 such as a mutation in position corresponding to Tyr164, Arg167, Lys168, Asp171, Lys172, Glu174, Thr175, Phe176, Arg178, Ile179, Cys182, Cys189 and Gly190.
58. The growth hormone compound, according to any of the previous embodiments, wherein the growth hormone variant has at least one point mutation in Helix 4 such as a mutation in position corresponding to Lys172, Glu174, Thr175, Phe176, and Arg178.
59. The growth hormone compound, according to any of the previous embodiments, wherein the growth hormone variant has at least one point mutation in site 2 (N-term and Helix 1 (1-35) and Loop 2/Helix 3 (99-127)).
60. The growth hormone compound, according to any of the previous embodiments, wherein the growth hormone variant has at least one point mutation in N-term and Helix 1 such as a mutation in position corresponding to Phe1, Pro2, Ile4, Pro5, Leu6, Arg8, Leu9, Asn12, Ala13, Leu15, Arg16 and Arg19.
61. The growth hormone compound, according to any of the previous embodiments, wherein the growth hormone variant has at least one point mutation in Loop2/Helix3 such as a mutation in position corresponding to Val102, Tyr103, Asn109, Asp116, Leu117, Glu119, Gly120 and Thr123.
62. The growth hormone compound, according to any of the previous embodiments, wherein the growth hormone variant has at least one point mutation in Helix 3 (AA107-127).
63. The growth hormone compound, according to any of the previous embodiments, wherein the growth hormone variant has at least one point mutation in Helix 3 such as a mutation in position corresponding to Asn109, Asp116, Leu117, Glu119, Gly120 and Thr123.
64. The growth hormone compound, according to any of the previous embodiments, wherein the growth hormone variant has at least one point mutation in position Gly120.
65. The growth hormone compound, according to any of the previous embodiments, wherein the growth hormone variant has at least one point mutation in a position corresponding to Lys41, Arg64, Lys172 and/or Gly120.
66. The growth hormone compound, according to any of the previous embodiments, wherein the growth hormone variant has at least one point mutation in a position corresponding to Lys41, Arg64 and/or Lys172.
67. The growth hormone compound, according to any of the previous embodiments, wherein the growth hormone variant has a deletion on at least one amino acid residue in site 1 or site 2.
68. The growth hormone compound, according to any of the previous embodiments, wherein the Fc-domain is covalently linked to the growth hormone variant.
69. The growth hormone compound, according to any of the previous embodiments, wherein the compound is linked by a peptide bond.
70. The growth hormone compound, according to any of the previous embodiments, wherein the compound comprises a GH variant and Fc polypeptide fusion protein.
71. The growth hormone compound, according to any of the previous embodiments, wherein the compound comprises a GH variant and Fc polypeptide fusion protein and wherein said fusion protein includes a linker peptide, such as a GS-linker.
72. The growth hormone compound, according to any of the previous embodiments, wherein the compound comprises only one GH variant polypeptide and two Fc polypeptides (monovalent).
73. The growth hormone compound, according to any of the previous embodiments, wherein the compound comprises two GH variant polypeptides and two Fc polypeptides (bivalent).
74. The growth hormone compound, according to any of the previous embodiments, wherein the GH variant polypeptide is linked to the Fc-domain via the N-terminal or the C-terminal of GH.
75. The growth hormone compound, according to any of the previous embodiments, wherein the GH variant polypeptide is linked to the N-terminal or the C-terminal of the Fc polypeptide.
76. The growth hormone compound, according to any of the previous embodiments, wherein only one GH variant polypeptide is linked to the N-terminal of one Fc polypeptide of the Fc-domain.
77. The growth hormone compound, according to any of the previous embodiments, wherein only one GH variant polypeptide is linked to the C-terminal of one Fc polypeptide of the Fc-domain.
78. The growth hormone compound, according to any of the previous embodiments, wherein the Fc polypeptides of the Fc-domain are covalently linked.
79. The growth hormone compound, according to any of the previous embodiments, wherein the Fc polypeptides of the Fc-domain are covalently linked via at least one di-sulfide bond.
80. The growth hormone compound, according to any of the previous embodiments, wherein the Fc polypeptides are covalently linked via at least one di-sulfide bond in the hinge region.
81. The growth hormone compound, according to any of the previous embodiments, wherein the Fc polypeptides comprise two or three constant domains.
82. The growth hormone compound, according to any of the previous embodiments, wherein the Fc polypeptides are of isotype IgG, IgA or IgD.
83. The growth hormone compound, according to any of the previous embodiments, wherein the Fc polypeptides are of isotype IgG, with subtype IgG1, IgG2 or IgG4, such as IgG1 as defined by SEQ ID NO 3 (11-227).
84. The growth hormone compound, according to any of the previous embodiments, wherein the Fc polypeptides comprise a hinge region and wherein said hinge region optionally comprises one or more cysteines.
85. The growth hormone compound, according to any of the previous embodiments, wherein the Fc polypeptides has one or more point mutations.
86. The growth hormone compound, according to any of the previous embodiments, wherein the Fc polypeptides are of the IgG4 type and have a proline residue in position S228.
87. The growth hormone compound, according to any of the previous embodiments, wherein the Fc polypeptides are of the IgG1 type and have one or more of the L234A, L235E, and G237A point mutations.
88. The growth hormone compound, according to any of the previous embodiments, wherein the Fc polypeptides are of the IgG1 type and have one or more of the A330S and P331S point mutations.
89. The growth hormone compound, according to any of the previous embodiments, wherein the Fc polypeptides has one or more mutation to improve binding affinity to FcRn.
90. The growth hormone compound, according to any of the previous embodiments, wherein the Fc polypeptides are of the IgG1 type and have one or more of the M428L and N434S point mutations.
91. A pharmaceutical composition comprising a growth hormone compound, according to any of the previous embodiments.
92. A growth hormone compound, according to any of the previous embodiments, for use in a method of treatment.
93. A growth hormone compound, according to any of the previous embodiments, for use in a method of treatment of GHD and/or AGHD.
94. A growth hormone compound, according to any of the previous embodiments, for use in a method of treatment of IBD or CD.
95. A method for treatment of GHD and/or AGHD comprising administering a therapeutic effective amount of a growth hormone compound, wherein the growth hormone compound is a growth hormone variant linked to an antibody Fc-domain, to an individual in need.
96. An isolated nucleotide sequence encoding a GH variant and Fc polypeptide fusion according to any of the embodiments 70-90.
97. An expression vector encoding a GH variant and Fc polypeptide fusion according to any of the embodiments 70-90.
98. A host cell expressing a GH variant and Fc polypeptide fusion according to any of the embodiments 70-90.
99. A method for preparing a growth hormone compound according to any of the previous embodiments, comprising the steps of:
   a) obtaining a host cell expressing a growth hormone variant polypeptide,
   b) obtaining a host cell expressing an Fc polypeptide capable of forming an Fc-domain by association of two Fc-polypeptides,
   c) expressing and purifying said growth hormone variant polypeptide and said Fc-domain formed by two Fc-polypeptides from said host cell(s),
   d) linking the growth hormone variant polypeptide and the Fc-domain and
   e) obtaining said growth hormone compound.
100. A method for preparing a growth hormone compound according to any of the previous embodiments 68-88, wherein the compound comprises a GH variant and Fc polypeptide fusion, comprising the steps of:
   a) obtaining a host cell (1) expressing a GH variant and Fc polypeptide fusion,
   b) optionally obtaining a host cell (2) expressing a Fc polypeptide, wherein said host cell (2) may be identical to host cell (1) of a),
   c) expressing and purifying said polypeptide(s) from said host cell(s) and
   d) obtaining a growth hormone compound said compound comprising a GH-variant-Fc polypeptide fusion.
101. A method for preparing a growth hormone compound with increased plasma half-life comprising including a growth hormone variant as defined in any of the above embodiments.
102. The method of embodiment 101, wherein the increase plasma half-life is obtained relative to an equivalent compound including the human growth hormone sequence (SEQ ID NO. 1).
103. The method of embodiment 101, wherein growth hormone compound includes a growth hormone variant and a protracting entity.
104. The method of embodiment 103, wherein the growth hormone compound comprises a growth hormone variant linked to an antibody Fc-domain as protracting entity.

### EXAMPLES

### A. General Method for Preparing a GH-Fc Compound

Growth hormone polypeptides (wild type and variants) and Fc fusions hereof may be expressed and purified as follows.

The gene coding for the growth hormone polypeptided is inserted recombinant into a plasmid vector. A suitable E. coli strain is subsequently transformed using the plasmid vector. hGH or GH variants may be expressed with an N-terminal methionine or as a MEAE fusion from which the MEAE sequence is subsequently cleaved off.

Site-specific point mutations have been introduced by using PCR (Quik Change Lightening Site-Directed Mutagenesis Kit, Agilent Technologies Site-Directed Mutagenesis Kit, Catalog #210519). By using recombinant nucleic acid techniques GH variants was fused with hIgG1 Fc with a S(G₄S)₆-linker between the two sequences. Genes encoding hGH-linker-Fc (hIgG1) and Fc (hIgG1) were cloned (separately) in individual pJSV002 plasmids (WO08009545) at EcoRI and BamHI sites, respectively, and transformed into E. coli Top10 for propagating to prepare plasmid. Fc variants may be obtained by introducing site-specific point mutations in the sequence encoding hIgG1 Fc.

Human and Rat Fc encoding DNA segments were used and fusions with human growth hormone or variants thereof where generated either C-terminal or N-terminal of the GH coding sequence. Here the peptide linker S(G₄S)₆ was inserted between GH part and Fc part. Likewise the attachment to Fc were also done either to the C-terminal or to the N-terminal e.g. N-terminal to the CH2 and CH3 regions maintaining part of the hinge region. The Fc sequence was shortened by deletion of “SEPKSC” while maintaining “DKTHTCPPCP” including two cysteines capable of stabilizing the Fc domain by di-sulfide bond (See SEQ ID NO.: 3).

### Cells and Expression of hGH and hGH Variant Polypeptides.

Cell stocks are prepared in 25% glycerol and stored at −80° C. Glycerol stock strains are inoculated into LB plates and subsequently incubated at 37° C. overnight. The content of each plate is washed with LB medium and diluted into 500 mL LB medium for expression. The cultures are incubated at 37° C. with shaking at 220 rpm until OD₆₀₀ 0.6 reached. Succeeding induction is performed using 0.2 mM IPTG at 25° C. for 16 hours. Cells are finally harvested by centrifugation.

Cells are subsequently suspended in 10 mM Tris-HCl, pH=9.0 containing 0.05% Tween 20, 2.5 mM EDTA, 4M urea, and optionally 10 mM cysteamine. The cells are disrupted using a cell disrupter at 30 kPSI. The supernatant is collected by centrifugation and subsequently subjected to chromatographic purification. The purification is performed using ion-exchange chromatography and hydrophobic interaction, followed by removal of the peptide tag using human dipeptidyl peptidase I (hDPPI) expressed from CHO cell. Final purification is achieved by isoprecipitation and ion-exchange chromatography. The purification can also be achieved by using but not limited to ion-exchange chromatography, hydrophobic interaction chromatography, affinity chromatography, size exclusion chromatography and membrane based separation techniques known to a person skilled in the art.

### Cells and Expression of Growth Hormone Fc-Fusions

HEK293 6E cells (Invitrogen) was grown to 1×10⁶ cells/ml in the medium of FreeStyle™-293 containing Geneticin 418, 25 ug/ml and pluronic F-68, 0.1% (Life Technologies). The two plasmids (pJSV002) encoding hGH-linker-Fc or Fc was transfected using Opti-MEMOI Reduced Serum Medium (Life Technologies) and 293fectin (Invitrogen). Each transfection used 100 ug of each plasmid for transfecting 200 ml HEK293 6E cells. After 1st day of growth, an equal volume of fresh medium was added to the culture, and culture supernatant was harvested after 4-5 more days of cultivation.

The supernatant of the culture was filtrated through 0.22 μm filter before uploaded onto a MabSelectSure affinity column (GE Healthcare). The target protein was eluted with buffer containing formic acid 20 mM, NaCl 100 mM, pH3.5. Peak fractions collected (monitored with UV280) were then adjusted to pH8.5 with Tris-HCl 1 M, pH9.0, and further purified with 30Q anion exchange chromatography (GE Healthcare), which was pre-equilibrated with buffer containing Tris-HCl 25 mM, pH8.5. The target protein was eluted with a 0 to 0.3 M linear gradient of NaCl in TrisHCl 25 mM, pH8.5 in 5 bed-volumes. The fractions containing the target protein was identified by SDS-PAGE, and pooled for buffer-exchanging into PBS, pH7.2 using a G-25 desalting column (GE Healthcare). Protein concentration was determined by measuring the absorbance at 280 nmol.

### Generation of Growth Hormone Conjugates

Conjugates of growth hormone polypeptides with a protracting moiety maybe performed as described in the art. The skilled person will know to apply various technologies adding the protracting moiety to different amino acid residues of the growth hormone polypeptide expressed and purified as described above. Examples of such methods can be found in references present in the background section refereeing to previously described methods of generating protracted growth hormone compounds.

### B. General Method for Testing Receptor Interaction of hGH and GH Compounds (Biacore) to hGHR Via Site 1

Receptor interaction of GH compounds is analysed using surface plasmon resonance (SPR) analysis. The method is general for the GH compounds.

The interaction of hGH and GH compounds with the hGH receptor via site 1 was studied by surface plasmon resonance using a Biacore T100 instrument (GE Healtcare, Sweden). Anti-hGH mAb (Fitzgerald Industries International, USA, #10G05B) was immobilized onto a CM-5 chip according to manufacturer's instruction at a level of typically 5000 RU. hGH or GH compounds are captured at 10-25 μg/ml in running buffer (10 mM HEPES, 0.15 M NaCl, 30 mM EDTA, 0.05% Surfactant P20, pH 7.4), which resulted in 250-400 RU captured ligand. hGHR at a concentration of 0-800 nmol was subsequently injected over the surface at 30 ml/min. A surface with immobilized anti-hGH mAb but without captured hGH was used as reference.

Kinetic data is analyzed with Biacore™ Evaluation Software 2.0 with the 1:1 Langmuir binding model.

### C. General Method for Testing Biological Activity of hGH and hGH Compounds (BAF Assay)

The biological activity of hGH compounds is measured in a cell based receptor potency proliferation assay, namely a BAF assay. The BAF-3 cells (a murine pro-B lymphoid cell line derived from the bone marrow) is IL-3 dependent for growth and survival. IL-3 activates JAK-2 and STAT which are the same mediators GH is activating upon stimulation. The BAF-3 cells were transfected with a plasmid containing the hGH receptor. Clones able to proliferate upon stimulation with hGH were turned into hGH-dependent cell lines hereafter referred to as BAF3-GHR. The cell lines respond to GH with a dose-related growth pattern and can therefore be used to evaluate the effect of different hGH compounds in a proliferation assay. The BAF-3GHR cells are grown in starvation medium (culture medium without GH) for 24 hours at 37° C., 5% CO2. The cells are centrifuged, the medium is removed and the cells are re-suspended in starvation medium to 2,22×105 cells/ml. Portions of 90 μl of the cell supernatant are seeded into microtiter plates (96 well NUNC-clone). Different concentrations of growth hormone compound are added to the cells, and the plates are incubated for 72 hours at 37° C., 5% CO2.

AlamarBlue is a redox indicator, AlamarBlue™ (BioSource cat no Dal 1025) which is reduced by reactions innate to cellular metabolism and, therefore, provides an indirect measure of viable cell number. The AlamarBlue™ is diluted 6 times (5 μl AlamarBlue™+25 μl stavation medium) and 30 μl of the diluted AlamarBlue™ is added to each well. The cells are then incubated for another 4 hours. Finally the metabolic activity of the cells is measure in a fluorescence plate reader using an excitation filter of 544 nmol and an emission filter of 590 nmol. The result for a given compound is expressed as the ratio between EC50 of said compound and the EC50 of wt hGH run in parallel.

### D. General Method for Evaluating Pharmacokinetics Parameters of Growth Hormone Compounds (Normal Rats)

The pharmacokinetic of the compounds of the examples is investigated in male Sprague Dawley rats after intravenous (i.v.) single dose administration.

Test compounds are diluted to a final concentration of 11 mg/mL in a dilution buffer consisting of: Glycine 20 mg/mL, mannitol 2 mg/mL, NaHCO₃ 2.5 mg/mL, pH adjusted to 8.2. The test compounds are studied in male Sprague Dawley rats weighing approximately 250 g. The test compounds are administered as a single injection either i.v. in the tail vein with a 27 G needle at a predetermined dose such as of 15 nmolol/rat in volume of 0.1 ml (concentration 150 nmolol/ml) or approximately 60 nmolol/kg body weight.

For each test compound blood sampling is conducted according to the following schedule:

| | Time (h) | | | | | | | | | | | | | |
|---|---|---|---|---|---|---|---|---|---|---|---|---|---|---|
| Animal | Predose | 0.08 | 0.5 | 1 | 2 | 4 | 8 | 18 | 24 | 48 | 96 | 168 | 240 | 336 |
| 1 | X | | | X | | | X | | | X | | | X | |
| 2 | X | | | X | | | X | | | X | | | X | |
| 3 | X | | | X | | | X | | | X | | | X | |
| 4 | | X | | | X | | | X | | | X | | | X |
| 5 | | X | | | X | | | X | | | X | | | X |
| 6 | | X | | | X | | | X | | | X | | | X |
| 7 | | | X | | | X | | | X | | | X | | |
| 8 | | | X | | | X | | | X | | | X | | |
| 9 | | | X | | | X | | | X | | | X | | |

At each sampling time 200 μl blood is drawn from the tail vein or the sublingual plexus using a 25 G needle. The blood is sampled into an EDTA coated test tube and stored on ice until centrifugation at 1200×G for 10 min at 4° C. Fifty μl plasma is transferred to a Micronic tube and stored at −20° C. until analysis.

Test substance concentrations will be determined by Luminescence Oxygen Channeling Immunoassay (LOCI), which is a homogenous bead based assay. LOCI reagents include two latex bead reagents and biotinylated GH binding protein, which is one part of the sandwich. One of the bead reagents is a generic reagent (donor beads) and is coated with streptavidin and contains a photosensitive dye. The second bead reagent (acceptor beads) is coated with an antibody making up the sandwich. During the assay the three reactants combine with analyst to form a bead-aggregate-immune complex. Illumination of the complex releases singlet oxygen from the donor beads which channels into the acceptor beads and triggers chemiluminescence which is measured in the EnVision plate reader. The amount of light generated is proportional to the concentration of hGH derivative. 2 μL 40× in LOCI buffer diluted sample/calibrator/control is applied in 384-well LOCI plates. 15 μL of a mixture of biotinylated GH binding protein and mAb M94169 anti-(hGH) conjugated acceptor-beads is added to each well (21-22° C.). The plates are incubated for 1 h at 21-22° C. 30 μL streptavidin coated donorbeads (67 μg/mL) is added to each well and all is incubated for 30 minutes at 21-22° C. The plates are read in an Envision plate reader at 21-22° C. with a filter having a bandwidth of 520-645 nmol after excitation by a 680 nmol laser. The total measurement time per well is 210 ms including a 70 ms excitation time. The limit of detection for growth hormone compounds is 50 pM. A non-compartmental pharmacokinetic analysis is performed on mean concentration-time profiles of each test compound using WinNonlin Professional (Pharsight Inc., Mountain View, Calif., USA). The pharmacokinetic parameter estimates of terminal half-life (t_{1/2}) and mean residence time (MRT) are calculated.

### E. Method for Evaluating In Vivo Response of Growth Hormone Compounds (Hypophysectomised Sprague Dawley Rats)

The in vivo response is studied in hypophysectomised male Sprague Dawley rats. The hypophysectomised rat is a well-known and recognised animal model of growth hormone deficiency, where no production of growth hormone occurs after the surgical removal of the pituitary gland. This also leads to low circulating levels of insulin-like growth factor-1 (IGF-1) another important clinical feature of growth hormone deficiency in humans.

The hypophysectomy is usually performed on 4 week old male rats weighing 90-100 g. The animals entering the study 3-4 weeks after the surgery weighing 100-110 g. Animals with a body weight gain of more than 10% during the 3-4 weeks after surgery are not allowed to enter the study.

### Hypophysectomy Procedure

### Anaesthesia and Pre-Operative Analgesia

The rats are anaesthetised with fentanyl-fluanisone (Hypnorm 0.315 mg fentanyl og 10 mg fluanisone per ml) and midazolam (Midazolam Accord 5 mg midazolam per ml). The rats are dosed i.p. 2 ml/kg with a mixture of fentanyl-fluanisone and midazolam diluted in sterile water. The resulting mixture contains 0.07875 mg fentanyl, 2.5 mg fluanisone and 1.25 midazolam per ml.

### Surgical Procedure

The rats are prepared for aseptic surgery. The rats are mounted in the Hoffman-Reiter stereotactic device designed for the hypophysectomy procedure.

An 18G needle on a glass syringe is introduced into the right ear of the rat. During a rotating movement the needle passes through the tympanic membrane, middle ear and temporal bone. From this position the pituitary gland is aspirated.

The rat is dismounted from the stereotactic device and transferred to a thermo plate for recovery. When the rat recovers it will be transferred to its cage.

### Post-Operative Analgesia and Care

Before recovery the rat is treated with carprofen s.c. (Rimadyl 50 mg carprofen per ml) 1 ml/kg with a solution containing 5 mg carprofen per ml diluted in sterile water. Post-operative analgesia is sustained for 2 days after surgery by adding 0.05 mg carprofen per ml to a 5% dextrose solution which is provided to the rat instead of drinking water. After the first 2 days post-surgery the rat will be provided with at 5% dextrose solution as drinking water for up to 10-14 days post-surgery.

Hypophysectomised Sprague Dawley rats were randomly allocated to different dosing groups with nine animals in each group. One group received vehicle only and served as an control group. In all test groups each animal received a single i.v. dose of 15 nmolol test compound. The body weight was measured at day 0, 1, 2, 3, 4, 7, 8, 9, 10, 11 and 14 between 8-10 am.

For each test compound blood sampling is conducted according to the following schedule:

| | Time (h) | | | | | | | | | | | | | |
|---|---|---|---|---|---|---|---|---|---|---|---|---|---|---|
| Animal | Predose | 0.08 | 0.5 | 1 | 2 | 4 | 8 | 18 | 24 | 48 | 96 | 168 | 240 | 336 |
| 1 | X | | | X | | | X | | | X | | | X | |
| 2 | X | | | X | | | X | | | X | | | X | |
| 3 | X | | | X | | | X | | | X | | | X | |
| 4 | | X | | | X | | | X | | | X | | | X |
| 5 | | X | | | X | | | X | | | X | | | X |
| 6 | | X | | | X | | | X | | | X | | | X |
| 7 | | | X | | | X | | | X | | | X | | |
| 8 | | | X | | | X | | | X | | | X | | |
| 9 | | | X | | | X | | | X | | | X | | |

At each sampling time 200 μl blood is drawn from the tail vein or the sublingual plexus using a 25 G needle. The blood is sampled into an EDTA coated test tube and stored on ice until centrifugation at 1200×G for 10 min at 4° C. 50 μl plasma is transferred to a Micronic tube and stored at −20° C. until analysis. Plasma concentration-time profiles are generated for each compound. A non-compartmental pharmacokinetic analysis is performed on mean concentration-time profiles of each test compound using WinNonlin Professional (Pharsight Inc., Mountain View, Calif., USA). The pharmacokinetic parameter estimates of terminal half-life (t½) and mean residence time (MRT) are calculated.

### F. Method for Detecting IGF Response in Rats

The plasma IGF-1 concentrations is determined by a commercial ELISA assay (Commercial assay from Immunodiagnostic Systems Ltd. Octeia Rat/Mouse IGF-1, Cat. no. AC-18F1 IDS Ltd., England). The assay is a sandwich ELISA using a highly IGF-1 specific polyclonal antibody as catcher, and a horseradish peroxidase labelled high affinity monoclonal antibody as detector. The assay lower limit of detection is 63 ng/ml. IGF-1 plasma concentration-time profiles are generated for each compound together with baseline corrected IGF-1 plasma concentration-time profiles. The time and extent the baseline corrected profile is above zero is used as a measure for the compound efficacy.

### G. Method for Evaluating Pharmacokinetics Parameters of Growth Hormone Compounds in Minipigs

The pharmacokinetic of the compounds of the examples is investigated in female Göttingen minipigs after subcutaneous (s.c.) single dose administration. Test compounds are diluted to a final concentration of 15 mg/mL in a dilution buffer consisting of: Glycine 20 mg/mL, mannitol 2 mg/mL, NaHCO3 2.5 mg/mL, pH adjusted to 8.2. The test compounds are studied in female Göttingen minipigs weighing approximately 10-12 kg.

The test compounds were administered as a single subcutaneous injection on the right side of the neck, approximately 5-7 cm from the ear and 7-9 cm from the middle of the neck. The injections were given with a stopper on the 21 G needle, allowing 0.5 cm of the needle to be introduced. Each animal received a dose of 20 nmolol/kg in a dosing volume of 0.1 mL/kg.

For each test compound blood sampling was conducted from each animal according to the following schedule: Predose, 1, 4, 12, 24, 36, 48, 72, 96, 168, 240, 336, 504, 672, 840, and 1008 hours after dosing. Blood samples of 2 ml were collected from unanaesthetised minipigs by use of Vacutainers inserted in V. Jugularis into EDTA tubes. Immediately after blood collection the tubes were inverted gently in order to ensure sufficient mixing. The blood was kept on ice for max. 10 minutes before centrifugation at 1500 g for ten minutes at 4° C. Two hundred μl plasma was pipetted into Micronic tubes for compound concentration determination, and 200 μl plasma was be pipetted into Micronic tubes for IGF-1 determination. The plasma samples were stored at −20° C. until analysis.

Test substance concentrations were determined by Luminescence Oxygen Channeling Immunoassay (LOCI), which is a homogenous bead based assay. LOCI reagents include two latex bead reagents and biotinylated GH binding protein, which is one part of the sandwich. One of the bead reagents is a generic reagent (donor beads) and is coated with streptavidin and contains a photosensitive dye. The second bead reagent (acceptor beads) is coated with an antibody making up the sandwich. During the assay the three reactants combine with analyst to form a bead-aggregate-immune complex. Illumination of the complex releases singlet oxygen from the donor beads which channels into the acceptor beads and triggers chemiluminescence which is measured in the EnVision plate reader. The amount of light generated is proportional to the concentration of hGH derivative. 2 μL 40× in LOCI buffer diluted sample/calibrator/control is applied in 384-well LOCI plates. 15 μL of a mixture of biotinylated GH binding protein and mAb M94169 anti-(hGH) conjugated acceptor-beads is added to each well (21-22° C.). The plates are incubated for 1 h at 21-22° C. 30 μL streptavidin coated donorbeads (67 μg/mL) is added to each well and all is incubated for 30 minutes at 21-22° C. The plates are read in an Envision plate reader at 21-22° C. with a filter having a bandwidth of 520-645 nmol after excitation by a 680 nmol laser. The total measurement time per well is 210 ms including a 70 ms excitation time. The limit of detection for growth hormone compounds is 50 pM.

A non-compartmental pharmacokinetic analysis was performed on mean concentration-time profiles of each test compound using WinNonlin Professional (Pharsight Inc., Mountain View, Calif., USA). The pharmacokinetic parameter estimates of terminal half-life (t½) and mean residence time (MRT) were calculated.

### Example 1

A series of compounds were prepared (as described above under C.) to compare the functionality of a human versus a rat Fc sequence and the position and valence of the growth hormone protein. The compounds were evaluated in normal rats as described above providing the pharmacokinetic data included in Table 1 below.

TABLE 1
Pharmacokinetic data for growth hormone compounds.

| | | | | T ½ | MRT |
|---|---|---|---|---|---|
| Growth | | Fc | | (hour) | (hour) |
| hor- | Linkage and | com- | | (from | (from |
| mone | valence | ponent | Linkage | rat i.v.) | rat i.v.) |
| WT | — | — | — | 0.23 | 0.15 |
| WT | Via C-term | Human | Via N-term of Fc | 3.5 | 6.2 |
| | Bivalent | IgG1 | (hinge s-s | | |
| | | | maintained) | | |
| WT | Via C-term | Rat IgG1 | Via N-term of Fc | 4.8 | 7.9 |
| | Bivalent | | (hinge s-s | | |
| | | | maintained) | | |
| WT | Via N-term | Human | Via C-term of Fc | 6.8 | 10.0 |
| | Monovalent | IgG1 | (hinge s-s | | |
| | | | maintained) | | |
| WT | Via C-term | Human | Via N-term of Fc | 6.1 | 10.6 |
| | Monovalent | IgG1 | (N-term hinge s-s | | |
| | | | maintained) | | |

The data obtained demonstrated that a pronounced increase in half-life (T½) and mean residence time (MRT) in rat is obtained for all compounds independent on linkage and Fc, while mono-valente with regards to growth hormone provides the highest values.

### Example 2

Further growth hormone fusion compounds were generated to evaluate any effect of using a growth hormone variant including up to four point mutations. The variant used includes one or more of the following point mutations K41A, R64A, K172A and G120R.

Pharmacokinetic data was obtained as described above and include in table 2 below.

TABLE 2
Pharmacokinetic data of growth hormone compounds. N.D (not determined)

| | | | Linkage | | |
|---|---|---|---|---|---|
| | Linkage and | Fc | (Hinge S-S | T ½ | MRT |
| Growth hormone | valence | component | maintained) | (hour) | (hour) |
| WT | Via C-term | Human IgG1 | Via N-term of Fc | 6.8 | 10.0 |
| | Monovalent | | | | |
| K41A, R64A, K172A | Via C-term | Human IgG1 | Via N-term of Fc | 45.5 | 49.0 |
| and G120R | Bivalent | | | | |
| K41A, R64A, K172A | Via N-term | Human IgG1 | Via C-term of Fc | 86.7 | 88.0 |
| and G120R | Monovalent | | | | |
| K41A, R64A, K172A | Via C-term | Human IgG1 | Via N-term of Fc | 103.4 | 123.9 |
| and G120R | Monovalent | | | | |
| K41A and R64A | Via C-term | Human IgG1 | Via N-term of Fc | 82.6 | 30.4 |
| | Monovalent | | | | |
| K41A and K172A | Via C-term | Human IgG1 | Via N-term of Fc | 82.5 | 38.8 |
| | Monovalent | | | | |
| R64A | Via C-term | Human IgG1 | Via N-term of Fc | 12.4 | 15.5 |
| | Monovalent | | | | |
| K172A | Via C-term | Human IgG1 | Via N-term of Fc | N.D. | N.D. |
| | Monovalent | | | | |

The data obtained demonstrated that a pronounced increase in half-life (T½) and mean residence time (MRT) is obtained for all compounds independent on linkage and Fc, while the compound with the four point mutations in growth hormone has the highest values in particular when attached monovalent via the C-terminal. Reducing the number of point mutations to either of K41A and K172A or K41A and R64A resulted in compounds still having a high T½ and MRT.

### Example 3

Further growth hormone fusion compounds were generated to evaluate any effect of using a growth hormone variant including a stabilizing di-sulfide bond and up to four additional point mutations. The stabilizing disulfide bond is obtained by introducing Q84C and Y143C mutations which have previously been shown to increase stability/protease resistance of growth hormone compound. The effects on the pharmacokinetic profile on compounds combining Q84C and Y143C mutations with one or more of the above point mutations K41A, R64A, K172A and G120R where tested.

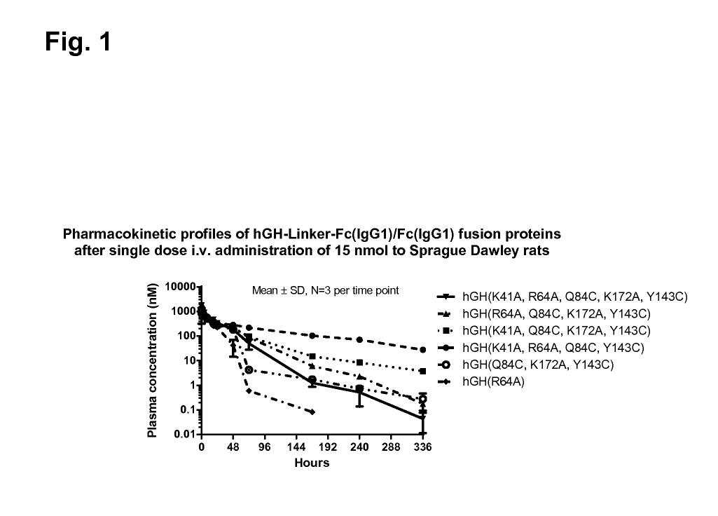

Pharmacokinetic data was obtained as described above and include in table 3 below and in FIG. 1.

TABLE 3
Pharmacokinetic data of growth hormone compounds.

| | GH | | | | | |
|---|---|---|---|---|---|---|
| | (Q84C | | | Linkage | | |
| Growth | and | Linkage and | | (Hinge S-S | T½ | MRT |
| hormone | Y143C) | valence | Fc component | maintained) | (hour) | (hour) |
| WT | Yes | Via C-term | Rat IgG1 | Via N-term of | 9.0 | 14.0 |
| | | Bivalent | | Fc | | |
| K41A, R64A, | No | Via C-term | Human IgG1 | Via N-term of | 103.4 | 123.9 |
| K172A and | | Monovalent | | Fc | | |
| G120R | | | | | | |
| K41A, R64A | Yes | Via C-term | Human IgG1 | Via N-term of | 90.4 | 118.7 |
| and K172A | | Monovalent | | Fc | | |
| R64A and | Yes | Via C-term | Human IgG1 | Via N-term of | 58.2 | 52.8 |
| K172A | | Monovalent | | Fc | | |
| K41A and | Yes | Via C-term | Human IgG1 | Via N-term of | 30.4 | 35.5 |
| K172A | | Monovalent | | Fc | | |
| K41A, and | Yes | Via C-term | Human IgG1 | Via N-term of | 27.0 | 25.4 |
| R64A | | Monovalent | | Fc | | |
| K172A | Yes | Via C-term | Human IgG1 | Via N-term of | 61.1 | 25.9 |
| | | Monovalent | | Fc | | |
| R64A | Yes | Via C-term | Human IgG1 | Via N-term of | 93.0 | 24.7 |
| | | Monovalent | | Fc | | |
| K41A and | Yes | Via C-term | Human IgG1 | Via N-term of | 66.0 | 25.6 |
| K172A | | Monovalent | (M428L and | Fc | | |
| | | | N434S) | | | |

Growth hormone variants including the additional disulfide bond and one or more point mutations selected from K41A, R64A and K172A have an increased half-life (T½) and mean residence time as did the variants not including the additional disulfide bond.

### Example 4

A selection of the compounds described above was tested in a BAF assay and the in vitro biological activity of the GH fusion compounds was evaluated relative to wild type human growth hormone.

TABLE 4
In vitro activities of growth hormone compounds.

| | GH | | | Linkage | hGHR (via | BAF |
|---|---|---|---|---|---|---|
| Growth | (Q84C and | Linkage and | Fc | (Hinge S-S | site 1) SPR | ratio |
| hormone | Y143C) | valence | component | maintained) | K_{D} (nmol) | X/hGH |
| K41A, R64A | Yes | Via C-term | Human | Via N-term of | 4150 | 199 |
| and K172A | | Monovalent | IgG1 | Fc) | | |
| R64A and | Yes | Via C-term | Human | Via N-term of | 969 | 6.6 |
| K172A | | Monovalent | IgG1 | Fc | | |
| K41A and | Yes | Via C-term | Human | Via N-term of | 235 | 8.3 |
| K172A | | Monovalent | IgG1 | Fc | | |
| K41A, and | Yes | Via C-term | Human | Via N-term of | 92 | 6.2 |
| R64A | | Monovalent | IgG1 | Fc | | |
| K172A | No | Via C-term | Human | Via N-term of | N.D. | 6.5 |
| | | Monovalent | IgG1 | Fc | | |
| WT | No | Via C-term | Human | Via N-term of | 0.43 | 2.3 |
| | | Monovalent | IgG1 | Fc | | |

N.D (not determined)

The GH fusion compound including the K41A, R64A and K172A mutations have high BAF ratio and a SPR K_{D} above 400 nmol to hGHR via site 1, confirming that the mutations influence the ability of the compound to interact with the receptor. It is further noted that the GH fusion variants that include only two of the mutations have a stronger interaction with the receptor and also a low BAF ratio demonstrating that the compounds are more likely to have biological activity.

### Example 5

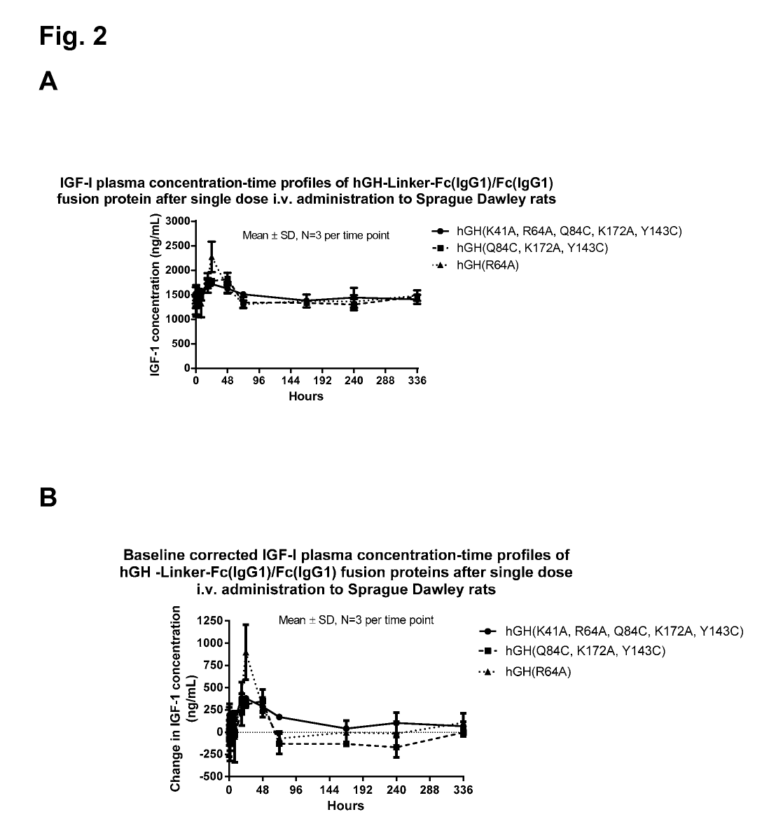

In further experiments the functionality of GH fusions were tested in Sprague Dawley rats. The IGF-1 plasma concentration was measured as described in Method F. The results (as shown in FIG. 2) demonstrate that the fusion compounds are capable of inducing and IGF-1 response in rat.

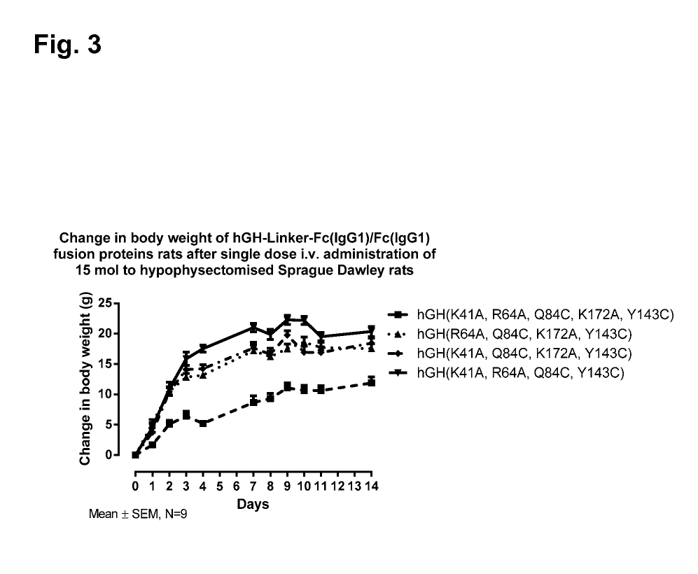

In addition, the effect on body weight was also measured and the results are shown in FIG. 3. As evident by the increased body weight of the animals and the observed IGF-1 response the GH fusion compounds were found to be biologically active.

### Example 6

The pharmacodynamic parameters of selected compounds were estimated using mini-pigs as described in Method G. As above the compounds were prepared as monovalent fusions linking the N-terminal of the Fc sequence to the C-terminal of the GH variant as indicated.

The obtained data are provided in table 5 below

TABLE 5
Pharmacodynamic parameters of selected compound based
on single dosage administration to minipigs.

| | GH | | | | |
|---|---|---|---|---|---|
| Growth | (Q84C and | Cmax | AUC | t½ | MRT |
| hormone | Y143C) | (nmolol/L) | (h * nmolol/L) | (h) | ((h) |
| K41A and | yes | 162 ± 14.4 | 17100 ± 2930 | 38.6 | 79.6 |
| R64A | | | | | |
| K172A | yes | 159 ± 10.3 | 17800 ± 4380 | 47.4 | 88.9 |
| K172A | no | 130 ± 13.8 | 8910 ± 1850 | 28.2 | 57.0 |
| R64A | yes | 150 ± 18.7 | 14800 ± 3630 | 34.5 | 70.8 |
| R64A | no | 138 ± 22.0 | 9910 ± 2070 | 20.5 | 56.2 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A growth hormone compound comprising a growth hormone variant linked to an antibody Fc-domain (GH-variant-Fc).

2. The growth hormone compound, according to claim 1, wherein the growth hormone variant has reduced affinity to the human growth hormone receptor (hGHR).

3. The growth hormone compound, according to claim 1, wherein the growth hormone compound binds hGHR via site 1 with an affinity (K_{D}) between 5000-0.5 nmol.

4. The growth hormone compound, according to claim 1, wherein the growth hormone compound has an increased in vivo half-life compared to the equivalent hGH-Fc compound.

5. The growth hormone compound, according to claim 1, wherein the growth hormone compound has an increased MRT compared to the equivalent hGH-Fc compound.

6. The growth hormone compound, according to claim 1, wherein the growth hormone compound is capable of inducing an extended IGF-1 response.

7. The growth hormone compound, according to claim 1, wherein the growth hormone compound induces an extended IGF-1 response, which lasts more than 24 hours.

8. The growth hormone compound, according claim 1, wherein the growth hormone compound induces weight gain in hypophysectomised rats.

9. The growth hormone compound, according to claim 1, wherein the growth hormone variant has at least one point mutation in a position corresponding to K41, R64 and/or K172 of human growth hormone (SEQ ID NO.: 1).

10. The growth hormone compound, according to claim 1, wherein the compound comprises one Fc-domain and one GH variant polypeptide (monovalent).

11. The growth hormone compound, according to claim 1, wherein the compound comprises at least one Fc polypeptide and GH variant (GH-variant-Fc polypeptide) fusion protein.

12. The growth hormone compound, according to claim 1, wherein the compound comprises at least one GH variant and Fc polypeptide (GH-variant-Fc polypeptide) fusion protein wherein the GH variant and Fc polypeptide are linked by a linker peptide.

13. The growth hormone compound, according to claim 1, wherein the Fc-polypeptide comprises a hinge region including one or more cysteines.

14. A method of treating GHD, AGHD, IBD or CD, comprising administering the compound of claim 1 to a subject in need thereof.

15. An expression vector encoding the GH variant and Fc polypeptide fusion according to claim 12.

16. A host cell expressing the GH variant and Fc polypeptide fusion according to claim 12.

17. The growth hormone compound according to claim 3, wherein the growth hormone compound binds hGHR via site 1 with an affinity (K_{D}) of between 4000-1.0 nmol, between 2500-10 nmol, between 1000-25 nmol, between 500-50 nmol, or between 250-75 nmol.

18. The growth hormone compound according to claim 7, wherein the growth hormone compound induces an extended IGF-1 response, which lasts more than 48 hours, more than 96 hours or more than 144 hours.

19. The growth hormone compound according to claim 12, wherein the linker peptide is a GS linker.
